(19) United States
(12) Patent Application Publication
(10) Pub. No.: US 2004/0215284 A1
(43) Pub. Date: Oct. 28, 2004

(54) Treatment of neuromuscular dysfunction of the lower urinary tract with selective mGlu5 antagonists

(75) Inventors: Amedeo Leonardi, Milan (IT); Rodolfo Testa, Vignate (IT); Elena Poggesi, Milano (IT)

Correspondence Address: DARBY & DARBY P.C., P. O. BOX 5257, NEW YORK, NY, 10150-5257, US

(73) Assignee: Recordati S.A.

(21) Appl. No.: 10/768,953

(22) Filed: Jan. 29, 2004

Related U.S. Application Data
(60) Provisional application No. 60/506,631, filed on Sep. 26, 2003.

(30) Foreign Application Priority Data
Jan. 30, 2003 (IT) MI2003A 000151

(51) Int. Cl. A61N 1/18

(52) U.S. Cl. 607/40

(57) ABSTRACT

The invention is directed to methods of using antagonists selective for the metabotropic mGlu5 receptor to treat conditions of neuromuscular dysfunction of the lower urinary tract in a mammal. Provided are methods of treating a mammal suffering from a condition of neuromuscular dysfunction of the lower urinary tract by administering a selective mGlu5 antagonist. The selective mGlu5 antagonist may be administered alone or in combination with one or more additional therapeutic agent for treating such a condition. Also provided are methods of identifying selective mGlu5 antagonists that are useful for treating neuromuscular dysfunction of the lower urinary tract in a mammal.

### CROSS REFERENCE TO RELATED APPLICATIONS

This application claims priority under 35 U.S.C. § 119(e) of provisional application No. 60/506,631, filed Sep. 26, 2003, and under 35 U.S.C. § 119(a)-(d) of Italian patent application no. MI 2003 A 000151, filed Jan. 30, 2003. Each of the foregoing applications is hereby incorporated herein by reference in its entirety.

### FIELD OF THE INVENTION

This invention relates to compounds having selective affinity for the mGlu5 subtype of metabotropic glutamate receptors, pharmaceutical compositions thereof and uses for such compounds and compositions.

### BACKGROUND OF THE INVENTION

In mammals, micturition is a complex process that requires the integrated actions of the bladder, its internal and external sphincters, and the musculature of the pelvic floor. Neurological control over these muscles occurs at three levels—in the bladder wall or sphincters, in the autonomic ganglia of the spinal cord, and in the central nervous system in the pontine micturition center (PMC) of the medulla oblongata (pons), under the control of the cerebral cortex (De Groat, Neurobiology of Incontinence, Ciba Foundation Symposium 151:27, 1990).

Micturition results from contraction of the bladder detrusor muscle, which consists of interlacing smooth muscle fibers that are under parasympathetic autonomic control from the sacral spinal cord. A simple voiding reflex is formed by sensory nerves for pain, temperature, and distension that run from the bladder to the sacral cord. However, sensory tracts from the bladder also reach the pontine micturition center, resulting in the generation of nerve impulses that normally act at the spinal cord to suppress the sacral spinal reflex arc controlling bladder emptying. As a result, normal micturition is initiated by voluntary suppression of the cortical inhibition of the reflex arc and by relaxation of the muscles of the pelvic floor and the external sphincter. These events are followed by contraction of the detrusor muscle and voiding.

Functional abnormalities of the lower urinary tract, e.g., dysuria, incontinence, and enuresis, are common in the general population. Dysuria includes urinary frequency, nocturia, and urgency, and may be caused by cystitis (including interstitial cystitis), prostatitis or benign prostatic hyperplasia (BPH), which affects about 70% of elderly males, or by neurological disorders. Incontinence syndromes include stress incontinence, urgency incontinence, overflow incontinence, and mixed incontinences. Enuresis refers to the involuntary passage of urine at night or during sleep.

Treatment of neuromuscular disorders of the lower urinary tract has typically involved administration of compounds that act directly on the bladder muscles, such as flavoxate, a spasmolytic drug (Ruffinan, J., Int. Med. Res. 16:317, 1988) that is also active on the PMC (Guarneri et al., Drugs of Today, 30:91, 1994), or anticholinergic compounds such as oxybutynin (Andersson, Drugs 36:477, 1988) and tolterodine (Nilvebrant, Life Sci. 68: 2549, 2001). The use of α1-adrenergic receptor antagonists for the treatment of BPH is also common (Lepor, Urology, 42:483, 1993). Treatment of lower urinary tract disorders with α1-adrenergic receptor antagonists, including subtype selective antagonists is described in U.S. Pat. Nos. 5,990,114; 6,306,861; 6,365,591; 6,387,909; and 6,403,594. Treatments that involve direct inhibition of the pelvic musculature, including the detrusor muscle, however, may have undesirable side effects, such as incomplete voiding, accommodation reflex paralysis, tachycardia and dry mouth (Andersson, Drugs 35:477, 1988). Thus, it would be preferable to treat neuromuscular disorders of the lower urinary tract with compounds that act via the peripheral or central nervous system, to affect, for example, the sacral spinal reflex arc and/or the inhibitory impulses of the pontine micturition center in a manner that restores normal functioning of the micturition mechanism.

Glutamic acid, an excitatory amino acid, is present at synapses throughout the central nervous system and is known to act on at least two types of receptors: ionotropic and metabotropic glutamate receptors.

Upon activation, ionotropic glutamate receptors form ligand-gated ion channels and, thereby, directly mediate electrical signalling of nerve cells, producing rapid and relatively large conductance changes in the post-synaptic membranes. Metabotropic glutamate receptors (mGluRs) regulate electrical signalling indirectly, by influencing intracellular metabolic processes via G-proteins. Changes in the post-synaptic cell that are mediated through mGluRs are consequently relatively slow over time, compared to the rate at which effects are mediated through ionotropic glutamate receptors, and are not linked to rapid and large changes in neuronal membrane conductance.

Three subtypes of ionotropic glutamate receptors have been described, i.e., the NMDA, AMPA and kainate subtypes.

Eight subtypes of metabotropic glutamate receptors have been cloned. The subtypes are classified into three groups on the basis of sequence similarities, and pharmacological and biochemical properties (Spooren et al., Trends Pharmacol. Sci. 22: 331-337, 2001): Group I mGluRs (mGlu1 and mGlu5), Group II mGluRs (mGlu2 and mGlu3) and Group III mGluRs (mGlu4, mGlu6, mGlu7 and mGlu8). Alternative splicing is a source of receptor diversity among Group I and Group III receptors.

The structure of mGluRs is reviewed in Hermans and Challis (Biochem. J. 359: 465-484, 2001). With regard to Group I, mGlu1 receptors in rat comprise four isotypes, mGlu1a, mGlu1b, mGlu1c and mGlu1d, comprising 1199, 906, 897 and 912 amino acids, respectively. The isotypes share a common N-terminal domain of 887 amino acids joined respectively to a C-terminal domain of 312 (mGlu1a), 19 (mGlu1b), 10 (mGlu1c) and 25 (mGlu1d) amino acids. The differing C-terminal domains are generated by alternative splicing. Similarly, there are three known human mGlu1 receptors, mGlu1a (1194 amino acids), mGlu1b (906 amino acids) and mGlu1d (908 amino acids). The human receptors comprise a common N-terminal domain of 887 amino acids, joined respectively to C-terminal domains of 307 (mGlu1a), 19 (mGlu1b) and 21 (mGlu1d) amino acids that are generated by alternative splicing.

With respect to function, in all isoforms from either rat or human, the common N-terminal domain comprises, in order from the N-terminus, an extracellular domain, a typical G-protein coupled receptor transmembrane domain composed of seven transmembrane α-helices, and the beginning of an intracellular domain. The variable C-terminal domain forms a second intracellular loop that completes the intracellular domain of each isotype. Ligand specific recognition and binding takes place in the extracellular domain. G-protein binding is effected by the intracellular domain and intracellular loops connecting certain transmembrane α-helices. The ligand affinity and the ability to stimulate the second messenger cascade are the same for the four subtypes.

The known Group I mGlu5 receptors in both human and rat comprise mGlu5a and mGlu5b subtypes. In both human and rat, subtype mGlu5b differs from subtype mGlu5a in that mGlu5b includes a 32 amino acid insertion in the C-terminal intracellular domain, 50 residues downstream (i.e., towards the C-terminus) from the end of the transmembrane domain, that is not present in subtype mGlu5a. The difference between subtypes is due to alternative mRNA splicing. Accordingly, rat mGlu5a is 1171 amino acids and mGlu5b is 1203 amino acids, the difference being due to inclusion the 23 amino at positions 876-907 of the mGlu5b receptor. Human mGlu5a is 1180 amino acids and mGlu5b is 1212 amino acids, the difference being due to the 32 amino acids at positions 877-908 of the mGlu5b receptor. (Hermans et al., Biochem. J. 359: 465-484, 2001).

Regarding Group III receptors, alternative splicing gives rise to two isoforms respectively of mGlu4 and mGlu7 receceptors (i.e., subtypes “a” and “b”) and three isoforms of mGlu8 receptor (i.e., subtypes “a”, “b” and “c”) (Hermans et al., Biochem. J. 359: 465-484, 2001). mGlu6 receptor, by comparison, is currently known to have only a single isotype (Hermans et al., Biochem J. 359:465-484, 2001). Rat and human mGlu4a receptors are both 912 amino acids long. Rats also produce mGlu4b subtype that is 983 amino acids long, due to alternative splicing that replaces the last 64 amino acids of the mGlu4a subtype with a different set of 135 amino acids (Thomsen et al., Neuropharm., 36:21-30, 1997). Similarly, mGlu6, mGlu7a, mGlu7b, mGlu8a and mGlu8b have the same length in rat and man: 877, 915, 922, 908 and 908 amino acids, respectively. In both mGlu7 and mGlu8 receptors, the “b” isoform is due to a alternative splicing that results in a change of translation frame relative the “a” isoform and leads to replacement of the last 16 amino acids of mGlu7a and mGlu8a with a different set of 23 or 16 amino acids, respectively (Corti, et al., Eur. J. Neurosci., 10:3629-3641, 1998). The mGlu8c subtype was identified only in human and is only 501 amino acids long. An out-of-frame insertion generates a stop codon before the first alpha helix of the transmembrane domain; thus, the predicted human mGlu8c protein could represent a secreted isoform of receptor (Malherbe, et al., Brain Res. Mol. Brain Res., 67:201-210, 1999.)

Binding of glutamate to Group I receptors results in a G-protein-mediated activation of phospholipase C and breakdown of membrane phospholipids into the chemical messengers inositol trisphosphate and diacylglycerol. Inositol trisphosphate releases Ca²⁺ from internal stores. Consequently, activation of Group I mGluRs typically mediates excitation or increases the excitability of neurons.

Binding of glutamate at Group II or III receptors induces a G-protein-mediated inhibition of adenylate cyclase. Depressed adenylate cyclase activity results in reduced production of the cyclic adenosine monophosphate. Consequently, activation of Group II and III mGluRs typically mediates a depression of synaptic transmission.

The function of glutamatergic receptors in the micturition reflex pathway has been examined by administering drugs which block ionotropic glutamate receptors. Intravenous administration of MK 801, a non competitive blocker of the NMDA receptor, for example, decreased the amplitude of bladder contractions. MK 801 also increased the micturition volume threshold in some experiments, but not in others (deGroat, W. C., et al., In: Nervous control of the urogenital system, C. A. Maggi (ed.), Harwood Academic Publishers, pp. 227-290, 1993).

International patent publication number WO 00/63166 discloses tricyclic carbamic acid derivatives useful for the treatment of different diseases, including urinary incontinence. The derivatives are disclosed to be agonists or antagonists of Group I mGlu receptors with specificity for the mGlu1 receptor.

International patent publication number WO 01/32632 discloses pyrimidine derivatives useful for the treatment of different diseases, including urinary incontinence. The derivatives are disclosed as selective antagonists of the mGlu 1 receptor with at least 10-fold selectivity for the mGlu1 receptor over the mGlu 5 receptor.

International patent publication number WO 01/27070 discloses new bisarylacetamides useful for the treatment of urinary incontinence, among other conditions. The molecules are disclosed to be agonists or antagonists selective for the mGlu 1 receptor.

U.S. Pat. No. 6,369,222 discloses heterocycloazepinyl-pyrimidine derivatives useful for the treatment of urinary incontinence, among other conditions. The derivatives are disclosed to be antagonists of the mGlu 1 receptor.

The aforementioned applications and patent, therefore, disclose mGlu 1 receptor antagonists as useful for treating urinary incontinence. None of the references, however, provide experimental support for treatment of urinary incontinence, either in human patients or in an animal model for lower urinary tract disease.

Patients with lower urinary tract conditions often respond to certain classes or subclasses of therapeutic agents. Furthermore, patients may respond initially to a therapeutic agent, but become non-responsive to the agent overtime. Additionally, patients may exhibit undesirable side effects when therapeutic agents are administered in concentrations required to treat lower urinary tract conditions. These side effects may be reduced or overcome by changing treatment to a different therapeutic agent, or by administering lower dosages of two or more therapeutic agents to achieve a therapeutic effect, wherein one or more of the lower dosages would not be sufficient to have a therapeutic when the respective therapeutic agent is used in monotherapy.

Accordingly, one of ordinary skill in the art will appreciate a continuing need to identify new therapeutic agents in the treatment of lower urinary tract disease. The new therapeutics can be used to treat lower urinary tract disease as monotherapy. New therapeutics may be especially useful to treat patients who fail to respond to one or more alternative therapeutic agents, who have become non-responsive to an alternative therapeutic agent, or who would benefit from combination therapy with two or more therapeutic agents.

We have tested the activity of selective mGlu1 and selective mGlu5 antagonists, in a rat model useful to detect activity on the lower urinary tract. Surprisingly, good activity was found for antagonists selective for the mGlu5 receptor, whereas two commercially available antagonists selective for the mGlu1 receptor failed to exhibit an effect. An antagonist selective for Group II mGluRs also failed to exhibit an effect in the rat model. Given these results, selective mGlu5 antagonists can be an effective means to treat lower urinary tract disorders.

### SUMMARY OF THE INVENTION

The invention is based on the finding that selective mGlu5 antagonists are useful in the treatment of neuromuscular dysfunction of the lower urinary tract in mammals.

Thus, the invention provides methods for treating neuromuscular dysfunction of the lower urinary tract in mammals, including without limitation, dysuria, incontinence, and enuresis. The methods involve administering to an affected mammal in need of treatment of neuromuscular dysfunction of the lower urinary tract, an effective amount of a selective mGlu5 antagonist. In preferred embodiments, the selective mGlu5 antagonist has a structure represented by general formulas I to V below.

(1) Compounds of general formula I:

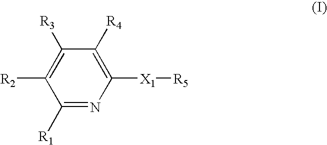

wherein:

R₁ represents hydrogen, lower alkyl, lower hydroxyalkyl, lower alkylamino, piperidino, carboxyl, esterified carboxyl, amidated carboxyl, lower alkoxy, lower haloalkyl, lower haloalkoxy, cyano, alkynyl, lower alkoxycarbonyl, di-(lower)alkylamino, lower alkylaminocarbonyl, trifluoromethylphenylaminocarbonyl or N-(lower)alkyl-N-phenylcarbamoyl, said N-(lower)alkyl and N-phenyl radicals being unsubstituted or substituted independently with a substituent selected from the group consisting of lower alkyl, lower alkoxy, halogen, and trifluoromethyl groups,

R₂ represents hydrogen, lower alkyl, carboxyl, esterified carboxyl, amidated carboxyl, lower hydroxyalkyl, hydroxyl, lower alkoxy or lower alkanoyloxy, lower alkoxycarbonyl, di-(lower)-alkylamino-(lower)alkanoyl, di-(lower)alkylaminomethyl, 4-(4-fluorobenzoyl)-piperidin-1-yl-carbonyl, 4-tert-butyloxycarbonylpiperazin-1-yl-carbonyl, 4-(4-azido-2-hydroxybenzoyl)-piperazin-1-yl-carbonyl or 4-(4-azido-2-hydroxy-3-iodo-benzoyl)-piperazin-1-yl-carbonyl,

R₃ represents hydrogen, lower alkyl, carboxy, lower alkoxycarbonyl, lower alkylcarbamoyl, lower hydroxyalkyl, di-(lower)alkylaminomethyl, morpholinocarbonyl or 4-(4-fluorobenzoyl)piperidin-1-yl-carbonyl,

R₄ represents hydrogen, lower alkyl, hydroxyl, lower hydroxyalkyl, lower aminoalkyl, (lower)alkylamino(lower)alkyl, di-(lower)-alkylamino(lower)alkyl, unsubstituted or hydroxy-substituted (lower)alkyleneamino(lower)alkyl, lower alkoxy, lower alkanoyloxy, lower aminoalkoxy, (lower)alkylamino(lower)alkoxy, di-(lower)-alkylamino(lower)alkoxy, lower alkoxycarbonyl, carboxy(lower)alkylcarbonyl, (lower)alkoxycarbonyl(lower)alkoxy, lower hydroxyalkyl, m-hydroxy-p-azidophenylcarbonylamino(lower)alkoxy, lower aminoalkoxy, phthalimido(lower)alkoxy, unsubstituted (lower)alkyleneamino(lower)alkoxy or (lower)alkyleneamino(lower)alkoxy substitued with hydroxyl or 2-oxo-imidazolidin-1-yl-groups, carboxyl, esterified carboxyl, amidated carboxyl, lower carboxyalkoxy or lower esterified carboxyalkoxy,

X₁ represents a lower alkenylene, lower haloalkenylene, lower alkynylene or lower haloalkynylene group, wherein each of the foregoing groups is linked via vicinal unsaturated carbon atoms, or an azo group (—N═N—), and

R₅ represents an aromatic or heteroaromatic group which is unsubstituted or substituted with one or more substituents selected from lower hydroxyalkyl, lower alkoxycarbonyl, lower alkanoyl, trifluoromethyl, trifluoromethoxy, trimethylsilylalkynyl, azido, lower aminoalkoxy, di-(lower)-alkylamino(lower)alkoxy, monohalobenzylamino, thienylmethylamino, thienylcarbonylamino, trifluoromethylphenylaminocarbonyl, tetrazolyl, lower alkanoylamino, benzylcarbonylamino, (lower)alkylaminocarbonylamino, (lower)alkoxycarbonylaminocarbonylamino, (lower)alkylsulfonyl, lower alkyl, halo, lower haloalkyl, lower haloalkoxy, lower alkenyl, lower alkynyl, unsubstituted phenyl or phenyl substituted with one or more substituent selected from the group consisting of lower alkyl, lower alkoxy, halo and trifluoromethyl groups, unsubstituted phenyl(lower)alkynyl or phenyl(lower)alkynyl substituted with one or more substituent selected from the group consisting of lower alkyl, lower alkoxy, halo and trifluoromethyl groups, hydroxyl, lower hydroxyalkyl, (lower)alkanoyloxy(lower)alkyl, lower alkoxy, lower alkenyloxy, lower alkylenedioxy, lower alkanoyloxy, lower amin alkoxy, (lower)alkylamino(lower)alkoxy, (lower)alkanoylamino(lower)alkoxy, N-(lower)-alkyl-N-(lower)-alkanoylamino(lower)alkoxy, unsubstituted phenoxy or phenoxy substituted with one or more substituent selected from the group consisting of lower alkyl, lower alkoxy, halo and trifluoromethyl groups, phenyl(lower)alkoxy or phenyl(lower)alkoxy wherein the phenyl group is substituted with one or more substituent selected from the group consisting of lower alkyl, lower alkoxy, halo and trifluoromethyl groups, acyl, carboxyl, esterified carboxyl, amidated carboxyl, cyano, carboxy(lower)alkylamino, esterified carboxy(lower)alkylamino, amidated carboxy(lower)alkylamino, phosphono(lower)alkylamino, esterified phosphono(lower)alkylamino, nitro, amino, lower alkylamino, di-(lower)-alkylamino, acylamino, N-acyl-N-(lower)-alkylamino, phenylamino, phenyl(lower)alkylamino, cycloalkyl(lower)alkylamino or heteroaryl(lower)alkylamino each of which may be unsubstituted or lower alkyl- lower alkoxy-, halo- and/or trifluoromethyl-substituted,

or an enantiomer, diastereoisomer, N-oxide, crystalline form, hydrate, solvate, pharmacologically active metabolite, prodrug, or pharmaceutically acceptable salt thereof.

Compounds of formula I having basic groups may form acid addition salts, and compounds of the formula I having acidic groups may form salts with bases. Compounds of formula I having basic groups and in addition having at least one acidic group, may also form internal salts.

Partial and total salts, i.e., salts with 1, 2 or 3, preferably 2, equivalents of base per mole of acid of formula I, or salts with 1, 2 or 3 equivalents, preferably 1 equivalent, of acid per mole of base of formula I are included within the present invention.

For the purposes of isolation or purification it is also possible to use pharmaceutically unacceptable salts. Only pharmaceutically acceptable, non-toxic salts are used therapeutically and they are therefore preferred.

When X₁ represents an alkenylene group, the trans configuration is preferred.

Preferred compounds of formula I are those wherein, either independently or together:

X₁ represents a (C₂₋₄)alkenylene, (C₂₋₄)haloalkenylene, (C₂₋₄)alkynylene or (C₂₋₄)haloalkynylene group, wherein each of the foregoing groups is linked via vicinal unsaturated carbon atoms,

R₁ is hydrogen, (C₁₋₄)alkyl, (C₁₋₄)alkoxy, (C₁₋₄)hydroxyalkyl, cyano, ethynyl, carboxy, (C₁₋₄)alkoxycarbonyl, di-(C₁₋₄)-alkylamino, (C₁₋₆)alkylaminocarbonyl, or trifluoromethylphenylaminocarbonyl,

R₂ is hydrogen, hydroxyl, (C₁₋₄)alkyl, (C₁₋₄)hydroxyalkyl, (C₁₋₄)alkoxy, carboxyl, (C₂₋₅)alkanoyloxy, (C₁₋₄)alkoxycarbonyl, di-(C₁₋₄)-alkylamino(C₁₋₄)alkanoyl, di-(C₁₋₄)alkylaminomethyl, 4-(4-fluorobenzoyl)-piperidin-1-yl-carbonyl, 4-tert-butyloxycarbonyl-piperazin-1-yl-carbonyl, 4-(4-azido-2-hydroxybenzoyl)-piperazin-1-yl-carbonyl, or 4-(4-azido-2-hydroxy-3-iodobenzoyl)-piperazin-1-yl-carbonyl,

R₃ is hydrogen, (C₁₋₄)alkyl, carboxyl, (C₁₋₄)alkoxycarbonyl, (C₁₋₄)alkylcarbamoyl, (C₁₋₄)hydroxyalkyl, di-(C₁₋₄)-alkylaminomethyl, morpholinocarbonyl or 4-(4-fluorobenzoyl)-piperidin-1-yl-carbonyl,

R₄ is hydrogen, hydroxyl, (C₁₋₄)alkoxy, carboxy, (C₂₋₅)alkanoyloxy, (C₁₋₄)alkoxycarbonyl, (C₁₋₄)aminoalkoxy, di-(C₁₋₄)-alkylamino(C₁₋₄)alkoxy, di-(C₁₋₄)-alkylamino(C₁₋₄)alkyl, carboxy(C₁₋₄)alkylcarbonyl, (C₁₋₄)alkoxycarbonyl(C₁₋₄)alkoxy, (C₁₋₄)hydroxyalkyl, di-(C₁₋₄)-alkylamino(C₁₋₄)alkoxy, or m-hydroxy-p-azidophenylcarbonylamino(C₁₋₄)alkoxy, and

R₅ is a group of formula

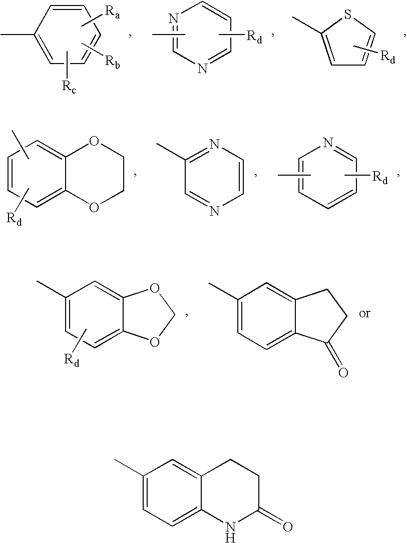

wherein

Rₐ and R_{b} independently of each other are hydrogen, hydroxyl, halo, nitro, cyano, carboxyl, (C₁₋₄)alkyl, (C₁₋₄)alkoxy, hydroxy(C₁₋₄)alkyl, (C₁₋₄)alkoxycarbonyl, (C₂₋₇)alkanoyl, (C₂₋₅)alkanoyloxy, (C₂₋₅)alkanoyloxy(C₁₋₄)alkyl, trifluoromethyl, trifluoromethoxy, trimethylsilylethynyl, (C₂₋₅)alkynyl, amino, azido, (C₁₋₄)aminoalkoxy, (C₂₋₅)alkanoylamino(C₁₋₄)alkoxy, (C₁₋₄)alkylamino(C₁₋₄)alkoxy, di-(C₁₋₄)-alkylamino(C₁₋₄)alkoxy, (C₁₋₄)alkylamino, di-(C₁₋₄)-alkylamino, monohalobenzylamino, thienylmethylamino, thienylcarbonylamino, trifluoromethylphenylaminocarbonyl, tetrazolyl, (C₂₋₅)alkanoylamino, benzylcarbonylamino, (C₁₋₄)alkylaminocarbonylamino (C₁₋₄)alkoxycarbonyl-aminocarbonylamino or (C₁₋₄)alkylsulfonyl,

R_{c} is hydrogen, fluoro, chloro, bromo, hydroxyl, (C₁₋₄)alkyl, (C₂₋₅)alkanoyloxy, (C₁₋₄)alkoxy or cyano, and R_{d} is hydrogen, halo or (C₁₋₄)alkyl.

More preferred compounds of formula I are those wherein X₁ is as defined above and

R₁ is hydrogen, (C₁₋₄)alkyl, (C₁₋₄)alkoxy, cyano, ethynyl or di-(C₁₋₄)-alkylamino,

R₂ is hydrogen, hydroxy, carboxy, (C₁₋₄)alkoxycarbonyl, di-(C₁₋₄)-alkylaminomethyl, 4-(4-fluoro-benzoyl)-piperidin-1-yl-carbonyl, 4-tert-butyloxycarbonyl-piperazin-1-yl-carbonyl, 4-(4-azido-2-hydroxybenzoyl)-piperazin-1-yl-carbonyl or 4-(4-azido-2-hydroxy-3-iodo-benzoyl)-piperazin-1-yl-carbonyl,

R₃ is as defined above,

R₄ is hydrogen, hydroxyl, carboxyl, (C₂₋₅)alkanoyloxy, (C₁₋₄)alkoxycarbonyl, amino(C₁₋₄)alkoxy, di-(C₁₋₄)-alkylamino(C₁₋₄)alkoxy, di-(C₁₋₄)-alkylamino(C₁₋₄)alkyl or hydroxy(C₁₋₄)alkyl, and

R₅ is a group of formula

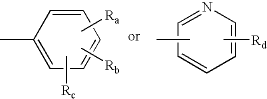

wherein

Rₐ and R_{b} independently of each other are hydrogen, halo, nitro, cyano, (C₁₋₄)alkyl, (C₁₋₄)alkoxy, trifluoromethyl, trifluoromethoxy or (C₂₋₅)alkynyl;

and R_{c} and R_{d} are as defined above.

More preferred are 2-methyl-6-(phenylethynyl)pyridine (MPEP) and 2-methyl-6-(2-phenylethenyl)pyridine (SIB 1893).

In certain embodiments, the invention provides compounds with a structure represented by general formula I-A.

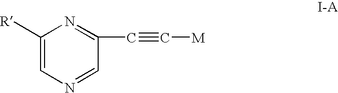

wherein

R′ is hydrogen or (C₁₋₄)alkyl and

M is a group of formula

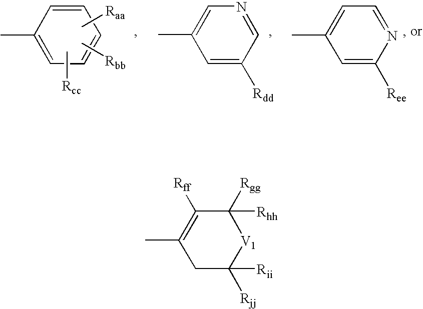

wherein

Rₐₐ, R_{bb} and R_{cc} are independently of each other hydrogen, (C₁₋₄)alkyl, (C₁₋₄)alkoxy, hydroxyl, (C₁₋₄)hydroxyalkyl, cyano or halo,

R_{dd} is cyano or halo,

Rₑₑ is hydroxyl, (C₁₋₄)alkyl or (C₁₋₄)alkoxy,

R_{ff} is hydrogen or (C₁₋₄)alkyl,

R_{gg} and Rₕₕ are hydrogen or together form a group of formula ═O, ═CH—CN, ═N—OH, ═N—O—(C₁₋₄)alkyl, ═CH—PO₃[(C₁₋₄)alkyl]₂ or ═CH—CO—Rₖₖ, wherein Rₖₖ is (C₁₋₄)alkoxy or —NRₗₗRₘₘ, where Rₗₗ and Rₘₘ are chosen independently from hydrogen, (C₁₋₄)alkyl and phenyl,

Rᵢᵢ and Rⱼⱼ are independently hydrogen, (C₁₋₄)alkyl or phenyl, and

V₁ is (CH₂)ₙ, CHRₙₙ, wherein n is 1, 2 or 3, Rₙₙ is hydroxyl, (C₁₋₄)alkyl, (C₁₋₄)alkoxy, (C₁₋₄)hydroxyalkyl, (C₁₋₄)alkoxy(C₁₋₄)alkyl, (C₁₋₄)alkoxycarbonyl, carbamoyl, (C₁₋₄)alkylcarbamoyl, phenyl, pyridyl, thienyl or (Rₒₒ, Rₚₚ)N-lower alkyl, wherein Rₒₒ is hydrogen, (C₁₄)alkyl, (C₁₋₄)alkanoyl or benzoyl and Rₚₚ is hydrogen or (C₁₋₄)alkyl, or, if R_{gg} and Rₕₕ are each hydrogen, Vₗ can also be NR_{qq}, wherein R_{qq} is (C₁₋₄)alkoxycarbonyl, benzyloxycarbonyl, benzoyl, thienyl, (C₁₋₄)alkanoyl, carbamoyl, mono- or di-(C₁₋₄)-alkylcarbamoyl or phenylcarbamoyl, any phenyl ring in R_{qq} being optionally substituted by one or more halo, cyano, (C₁₋₄)alkyl or (C₁₋₄)alkoxy groups,

or an enantiomer, diastereoisomer, N-oxide, crystalline form, hydrate, solvate, pharmacologically active metabolite, prodrug, or pharmaceutically acceptable salt thereof.

(2) Compounds Having a Structure Represented by General Formula II (i.e., II-A, II-B or II-C):

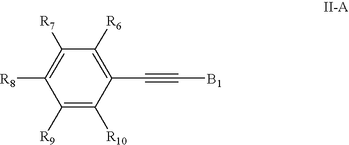

wherein

R₆, R₇, R₈, R₉ and R₁₀ represent, independently from each other, hydrogen, lower alkyl, lower alkoxy, —(CH₂)ₙ-halo, —(CH₂)_{n—NR}ₑR_{f}, —(CH₂)ₙ—N(Rₑ)—C(O)-(lower)alkyl, aryl or heteroaryl, which is unsubstituted or substituted by one or more lower alkyl groups;

B₁ represents

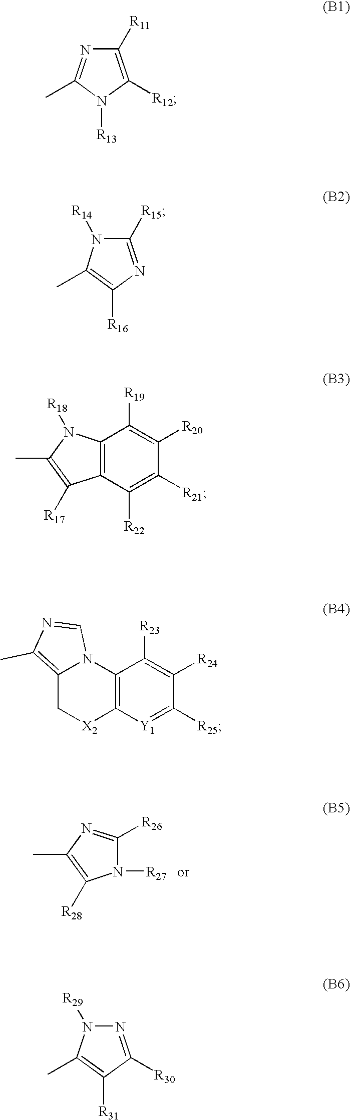

wherein

R₁₁ represents hydrogen, lower alkyl, —(CH₂)ₙ—C(O)ORₑ or halo;

R₁₂ represents hydrogen, lower alkyl, —(CH₂)ₙ—C(O)OR_{f}, halo, nitro or heteroaryl which is unsubstituted or substituted with lower alkyl or cycloalkyl;

R₁₃ represents hydrogen, lower alkyl, —(CH₂)ₙ—OH, —(CH₂)ₙ—C(O)OR_{g} or aryl;

R₁₄ represents lower alkyl;

R₁₅ represents hydrogen, lower alkyl or halo;

R₁₆ represents hydrogen or alkyl;

R₁₇ represents —(CH₂)ₙ—N(Rₑ)—C(O)-lower alkyl;

R₁₈ represents hydrogen or lower alkyl;

R₁₉, R₂₀, R₂₁ and R₂₂ represent, independently from each other, hydrogen, lower alkyl, —(CH₂)ₙ-halo or lower alkoxy;

R₂₃, R₂₄ and R₂₅ represent, independently from each other, hydrogen, lower alkyl, —(CH₂)ₙ-halo or lower alkoxy;

R₂₆ represents hydrogen or lower alkyl;

R₂₇ represents hydrogen, lower alkyl or lower alkyl substituted with one or more substituents selected from hydroxy and halo;

R₂₈ represents hydrogen, lower alkyl, lower alkanoyl or nitro;

R₂₉, R₃₀ and R₃₁ represent, independently from each other, hydrogen or lower alkyl;

Rₑ, R_{f} and R_{g} represent, independently from each other, hydrogen or lower alkyl;

n is 0, 1, 2, 3, 4, 5 or 6;

X₂ is —CH₂—, —O— or —S—; and

Y₁ is —CH═ or —N═;

or an enantiomer, diastereoisomer, N-oxide, crystalline form, hydrate, solvate, pharmacologically active metabolite, prodrug, or pharmaceutically acceptable salt thereof.

Examples of Compounds Within Formula II-A Are

1-methyl-2-phenylethynyl-1H-imidazole,

1-methyl-2-(4-methoxy-phenylethynyl)-1H-imidazole,

1-methyl-5-phenylethynyl-1H-imidazole, and

1-methyl-4-phenylethynyl-1H-imidazole, which may be either included or excluded from formula II.

In certain embodiments, the invention also relates to compounds of general formula II-B and II-C:

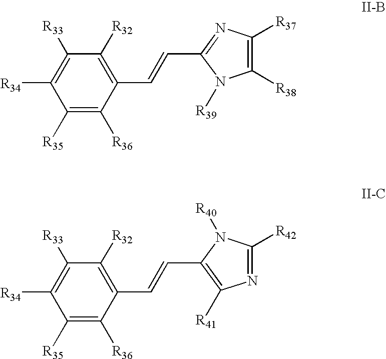

wherein

R₃₂, R₃₃, R₃₄, R₃₅ and R₃₆ represent, independently from each other, hydrogen, lower alkyl, —(CH₂)ₙ-halogen, lower alkoxy, —(CH₂)ₙ—NRₑR_{f}, —(CH₂)ₙ—N(Rₑ)—C(O)-(lower)alkyl, aryl or heteroaryl which is unsubstituted or substituted by one or more lower alkyl residues;

R₃₇ represents hydrogen, lower alkyl, —(CH₂)ₙ—C(O)ORₑ or halogen;

R₃₈ represents hydrogen, lower alkyl, —(CH₂)ₙ—C(O)OR_{f}, halogen, nitro or heteroaryl which is unsubstituted or substituted with lower alkyl or cycloalkyl;

R₃₉ represents hydrogen, lower alkyl, —(CH₂)ₙ—OH, —(CH₂)ₙ—C(O)OR_{g} or aryl;

R₄₀ represents lower alkyl;

R₄₁ represents hydrogen, halogen or lower alkyl; and

R₄₂ represents hydrogen or alkyl;

Rₑ, R_{f} and R_{g} represent, independently from each other, hydrogen or lower alkyl; and

and n=0, 1, 2, 3, 4, 5, or 6,

or an enantiomer, diastereoisomer, N-oxide, crystalline form, hydrate, solvate, pharmacologically active metabolite, prodrug, or pharmaceutically acceptable salt thereof.

Preferred compounds of formula II are compounds of formula II-A in which B₁ represents B1. Examples of such compounds are, without limitation,

1-methyl -2-phenylethynyl-1H-imidazole,

2-(5-nitro-2-phenylethynyl-imidazol-1-yl)-ethanol,

2-phenylethynyl-1H-imidazole,

5-(3,5-dimethyl-2-phenylethynyl-3H-imidazol-4-yl)-3-methyl-[1,2,4]oxadiazole, and

3-cyclopropyl-5-(3,5-dimethyl-2-phenylethynyl-3H-imidazol-4-yl)-[1,2,4]oxadiazole.

Also preferred compounds of formula II are compounds of formula II-A in which B₁ represents B2, such as, for example and without limitation, 1-methyl-5-phenylethynyl-1H-imidazole.

Also preferred compounds of formula II are compounds of formula II-A in which B₁ represents B3, such as, for example and without limitation, N-[2-(5-methoxy-2-phenylethynyl-1H-indol-3-yl)-ethyl]acetamide.

Also preferred compounds of formula II are compounds of formula II-A in which B₁ represents B4. Examples of such compounds are, without limitation,

3-phenylethynyl-4H-5-thia -2,6,9b-triaza-cyclopenta[a]naphthalene and

3-phenylethynyl-4H-5-oxa-2,9b-diaza-cyclopenta[a]naphthalene.

Also preferred compounds of formula II are compounds of formula II-A in which B₁ represents B5. Examples of such compounds are, without limitation,

1-chloro-3-(2-methyl -5-nitro-4-phenylethynyl -imidazol-l-yl)-propan-2-ol,

4-phenylethynyl-1H-imidazole,

1-methyl-4-phenylethynyl-1H-imidazole and

1,2-dimethyl-5-nitro-4-phenylethynyl -1H-imidazole.

Also preferred compounds of formula II are compounds of formula II-A in which B₁ represents B6, such as, for example and without limitation, 1,3-dimethyl-5-phenylethynyl-1H-pyrazole.

More preferred are compounds of formula II-A, in which B₁ represents B1 and R₁₂ represents (CH₂)ₙ—C(O)OR_{f} or heteroaryl which is unsubstituted or substituted by lower alkyl or cycloalkyl. Especially preferred are those, in which R₁₂ represents (CH₂)ₙ—C(O)OR_{f}, wherein n is 0 and R_{f} is lower alkyl.

(3) Compounds with a Structure Represented by General Formula III:

A₁—L₁—B₂ III

wherein

A₁ is a 5-, 6- or 7-membered ring having the structure:

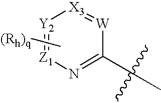

wherein

at least one of W, X₃, Y₂ and Z₁ is a group (CRₕ)ₚ, wherein p is 1 or 2; and the remainder of W, X₃, Y₂ and Z₁ are each independently O, N or S;

each Rₕ is independently, halogen, substituted or unsubstituted hydrocarbyl, substituted or unsubstituted aryl, substituted or unsubstituted heterocyclic, substituted or unsubstituted lower alkoxy, (lower)alkylcarbonyloxy, carboxyl, esterified carboxyl, amidated carboxyl, substituted or unsubstituted lower alkylthio, substituted or unsubstituted cycloalkyl, mercapto, nitro, carboxyl, carbamate, carboxamide, hydroxyl, ester, cyano, amine, amide, amidine, amido, sulfonyl, sulfonamide or N-(lower)-alkyl-N-phenylcarbamoyl wherein each nitrogen atom is independently unsubstituted or substituted independently with lower alkyl, lower alkoxy, halo or trifluoromethyl and wherein q is 0, 1, 2 or 3;

L₁ is substituted or unsubstituted alkenyl, alkynyl, or azo; and

B₂ is substituted or unsubstituted hydrocarbyl, substituted or unsubstituted cyclohydrocarbyl, substituted or unsubstituted heterocyclic, optionally containing one or more double bonds, or substituted or unsubstituted aryl,

wherein “substituted” refers to a radical wherein one or more hydrogen atoms has been replaced with a substituent selected from the group consisting of hydroxyl, alkyl, alkoxy, mercapto, aryl, heterocycle, halogen, trifluoromethyl, pentafluoroethyl, cyano, cyanomethyl, nitro, amino, N-substituted- or N,N-di-substituted amino, wherein one or both nitrogen atoms are substituted independently with alkyl, heterocycle, aryl which are each optionally further substituted independently with hydroxyl, alkyl or heterocycle, or, alkylamide, amidine, amido, carboxy, esterified carboxy, amidated carboxy, carboxamide, carbamate, ester, sulfonyl and sulfonamide groups, and the like,

or an enantiomer, diastereoisomer, N-oxide, crystalline form, hydrate, solvate, pharmacologically active metabolite, prodrug, or pharmaceutically acceptable salt thereof.

In accordance with certain embodiments of the present invention, A₁ is a 5-, 6- or 7-membered unsaturated heterocyclic group, containing a ring having at least one nitrogen atom located on the ring in a position adjacent to a carbon atom which bears a linking group as a substituent. The ring further contains 3, 4 or 5 independently variable atoms selected from carbon, nitrogen, sulfur and oxygen. Thus, A₁ can be pyridinyl, imidazolyl, pyridazinyl, pyrimidinyl, pyrazolyl, pyrazinyl, triazolyl, triazinyl, tetrazolyl, tetrazinyl, isoxazolyl, oxazolyl, oxadiazolyl, oxatriazolyl, oxadiazinyl, isothiazolyl, thiazolyl, dioxazolyl, oxathiazolyl, oxathiazinyl, azepinyl, diazepinyl, and the like. Those of skill in the art will recognize that multiple isomers exist for a single chemical formula; each of the possible isomeric forms of the various empirical formulae set forth herein are contemplated by the invention. When a variable ring atom is carbon, it bears a hydrogen, or is optionally substituted with halogen, substituted or unsubstituted hydrocarbyl, substituted or unsubstituted aryl, thiol, nitro, carboxyl, ester, cyano, amine, amide, carboxamide, amidine, amido, sulfonamide, and the like, with preferred embodiments having no substituent (i.e., q is 0) or bearing the following substituents: halogen, (C₁₋₄)alkyl, (C₁₋₄)fluoroalkyl, aryl, and amine. Substitution at position Z₁ of the ring is also preferred.

According to certain embodimentsof the invention, A₁ is a 5-, 6- or 7-membered ring containing, as ring members, a nitrogen atom and a sulfur atom. Groups contemplated for use by this embodiment of the invention include those wherein A₁ is isothiazol-3-yl(1,2-thiazol-3-yl), thiazol-4-yl(1,3,-thiazol-4-yl), thiazol-2-yl(1,3-thiazol-2-yl), 1,2-thiazin-3-yl, 1,3-thiazin-4-yl, 1,4-thiazin-3-yl, 1,3-thiazin-2-yl, thiazepinyl, and the like. Preferred groups include those wherein A₁ is isothiazol-3-yl(1,2-thiazol-3-yl), thiazol-4-yl(1,3-thiazol-4-yl) and thiazol-2-yl(1,3-thiazol-2-yl).

According to other embodiments of the invention, A₁ is a 5-, 6- or 7-membered ring containing, as ring members, a nitrogen atom and an oxygen atom. Groups contemplated by this embodiment of the invention include those wherein A₁ is 1,2-oxazin-3-yl, 1,3-oxazin-4-yl, 1,4-oxazin-3-yl, 1,3-oxazin-2-yl, oxazol-2-yl, isoxazol-3-yl, oxazol-4-yl, oxazepinyl, and the like. Preferred groups include those wherein A₁ is oxazol-2-yl, isoxazol-3-yl and oxazol-4-yl.

In still other embodiments of the invention, A₁ is a 5-, 6- or 7-membered ring containing, as a ring member, a nitrogen atom. Groups contemplated by these embodimens include those wherein A₁ is 2-pyridinyl and 2-pyrrolyl.

In still further embodiments of the invention, A₁ is a 5-, 6-, or 7-membered ring containing, as ring members, two nitrogen atoms. Groups contemplated by these embodiments include those wherein A₁ is 3-pyridazinyl(1,2-diazin-3-yl), pyrimidin-4-yl (1,3-diazin-4-yl), pyrazin-3-yl(1,4-diazin-3-yl), pyrimidin-2-yl(1,3-diazin-2-yl), pyrazol-3-yl(1,2-diazol-3-yl), imidazol-4-yl(1,3-isodiazol-4-yl), imidazol-2-yl (1,3-isodiazol-2-yl), diazepinyl, 1,3-isodiazol-4-yl, 1,3-isodiazol-2-yl and the like. Preferred groups include those wherein A₁ is 3-pyridazinyl (1,2-diazin-3-yl), pyrimidin-4-yl (1,3-diazin-4-yl), pyrazin-3-yl (1,4-diazin-3-yl), pyrimidin-2-yl (1,3-diazin-2-yl), 1,3-isodiazol-4-yl and 1,3-isodiazol-2-yl.

In accordance with still other embodiments of the invention, A₁ is a 5-, 6-, or 7-membered ring containing, as ring members, three nitrogen atoms. Groups contemplated by these embodiments include those wherein A₁ is 1,2,3-triazin-4-yl, 1,2,4-triazin-6-yl, 1,2,4-triazin-3-yl, 1,2,4-triazin-5-yl, 1,3,5-triazin-2-yl, 1,2,3-triazol-4-yl, 1,2,4-triazin-3-yl, triazepinyl, and the like. Preferred groups include those wherein A₁ is 1,2,3-triazin-4-yl, 1,2,4-triazin-6-yl, 1,2,4-triazin-3-yl, 1,2,4-triazin-5-yl, 1,3,5-triazin-2-yl, 1,2,3-triazol-4-yl, 1,2,4-triazol-3-yl.

In accordance with still other embodiments of the invention, A₁ is a 5-, 6-, or 7-membered ring containing, as ring members, four nitrogen atoms. Groups contemplated in these embodiments include those wherein A₁ is tetrazin-2-yl, tetrazin-3-yl, tetrazin-5-yl, tetrazolyl, tetrazepinyl, and the like. Preferred groups include those wherein A₁ is tetrazolyl.

In accordance with yet other embodiments of the invention, A₁ is a 5-, 6-, or 7-membered ring containing, as ring members, one sulfur atom and two nitrogen atoms. Groups contemplated by these embodiments include those wherein A₁ is 1,2,6-thiadiazin-3-yl, 1,2,5-thiadiazin-3-yl, 1,2,4-thiadiazin-3-yl, 1,2,5-thiadiazin-4-yl, 1,2,3-thiadiazin-4-yl, 1,3,4-thiadiazin-5-yl, 1,3,4-thiadiazin-2-yl, 1,2,4-thiadiazin-5-yl, 1,3,5-thiadiazin-4-yl, 1,3,5-thiadiazin-2-yl, 1,2,4-thiadiazol-3-yl, 1,2,3-thiadiazol-4-yl, 1,3,4-thiadiazol-2-yl, 1,2,5-thiadiazol-3-yl, 1,2,4-thiadiazol-5-yl, thiadiazepinyl, and the like. Preferred groups include those wherein A₁ is 1,2,4-thiadiazol-3-yl, 1,2,3-thiadiazol-4-yl, 1,3,4-thiadiazol-2-yl, 1,2,5-thiadiazol-3-yl and 1,2,4-thiadiazol-5-yl.

In accordance with yet other embodiments of the invention, A₁ is a 5-, 6-, or 7-membered ring containing, as ring members, one oxygen atom and two nitrogen atoms. Groups contemplated by these embodiments include those wherein A₁ is 1,2,6-oxadiazin-3-yl, 1,2,5-oxadiazin-3-yl, 1,2,4-oxadiazin-3-yl, 1,2,5-oxadiazin-4-yl, 1,2,3-oxadiazin-4-yl, 1,3,4-oxadiazin-5-yl, 1,3,4-oxadiazin-2-yl, 1,2,4-oxadiazin-5-yl, 1,3,5-oxadiazin-4-yl, 1,3,5-oxadiazin-2-yl, 1,2,4-oxadiazol-3-yl, 1,2,3-oxadiazol-4-yl, 1,3,4-oxadiazol-2-yl, 1,2,5-oxadiazol-3-yl, 1,2,4-oxadiazol-5-yl, oxadiazepinyl, and the like. Preferred groups include those wherein A₁ is 1,2,4-oxadiazol-3-yl, 1,2,3-oxadiazol-4-yl, 1,3,4-oxadiazol-2-yl, 1,2,5-oxadiazol-3-yl and 1,2,4-oxadiazol-5-yl.

In accordance with still another embodiment of the invention, A₁ is a 5-, 6-, or 7-membered ring containing as ring members, one up to six nitrogen atoms, and/or one up to six carbon atoms, and/or zero up to five sulfur atoms, and/or zero up to five oxygen atoms.

Further, in certain embodiments, L₁ is a linking group which links groups A₁ and B₂. L₁ is selected from substituted or unsubstituted alkenylene groups, alkynylene groups or azo groups. Preferred compounds are those wherein L₁ is alkenylene or alkynylene groups containing two carbon atoms, with alkynylene most preferred.

Further, in certain embodiments of the invention, B₂ is a group linked through bridging group L₁ to group A₁. Radicals contemplated for use in the invention are those wherein B₂ is substituted or unsubstituted hydrocarbyl, substituted or unsubstituted cyclohydrocarbyl, substituted or unsubstituted heterocycle, optionally containing one or more double bonds, substituted or unsubstituted aryl, and the like.

Preferred compounds are those wherein B₂ is a substituted or unsubstituted hydrocarbyl selected from substituted or unsubstituted alkyl groups, alkenyl groups, dialkenyl groups, trialkenyl groups, alkynyl groups, alkadiynyl groups, alkatriynyl groups, alkenynyl groups, alkadienynyl groups, alkenediynyl groups, and the like.

Further preferred are compounds wherein B₂ is a substituted or unsubstituted cyclohydrocarbyl selected from substituted or unsubstituted cycloalkyl groups, cycloalkenyl groups, cycloalkadienyl groups, cycloalkatrienyl groups, cycloalkynyl groups, cycloalkadiynyl groups, bicyclic hydrocarbon groups wherein two rings have two atoms in common, and the like. Especially preferred compounds are those wherein B₂ is cycloalkyl and cycloalkenyl having in the range of 4 up to about 8 carbon atoms. Exemplary compounds include cyclopropanyl, cyclopentenyl and cyclohexenyl. Also especially preferred are bicyclic hydrocarbon groups wherein two rings have two atoms in common; exemplary compounds include indenyl, dihydroindenyl, naphthalenyl and dihydronaphthalenyl.

Still fturther preferred are compounds wherein B₂ is a substituted or unsubstituted heterocycle, optionally containing one or more double bonds. Exemplary compounds include pyridyl, thiazolyl, furyl, dihydropyranyl, dihydrothiopyranyl, piperidinyl, isoxazolyl, pyridazinyl, pyrimidinyl, pyrazinyl, and the like. Also preferred are compounds wherein B₂ is substituted or substituted aryl. Especially preferred compounds are those wherein substituents are aryl and heterocycle, optionally bearing further substituents as described herein, methyl, trifluoromethyl, cyclopropyl, alkoxy, halogen and cyano. Also preferred are compounds wherein B₂ is a bicyclic heterocycle group wherein two rings have two atoms in common. Exemplary compounds include indolyl and isoquinolinyl.

Most preferred compounds of formula III are:

3-(2-methylthiazol-4-yl)ethynylpyridine (MTEP);

2-methyl-4-[(E)-2-phenylethenyl]-1,3-thiazole;

4-(2-pyrimidinylethynyl)isoquinoline;

2-(3,4-dihydro-2-naphthalenylethynyl)pyridine;

4,6-dimethyl-2-(phenylethynyl)pyrimidine; and

2-(1-cyclopenten-1-ylethynyl)-6-methylpyridine.

(4) Compounds with a structure represented by general formula IV

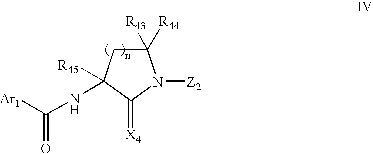

wherein,

n is 0, 1 or 2;

X₄ is O, S, NH, or NOH;

R₄₃ and R₄₄ are each independently hydrogen, CN, COORᵢ, CONHRᵢ, (C₁₋₆)alkyl, tetrazole, or R₄₃ and R₄₄ together represent an oxo group;

Rᵢ is hydrogen or (C₁₋₆)alkyl;

R₄₅ is (C₁₋₆)alkyl, (C₂₋₆)alkenyl, (C₃₋₈)cycloalkyl, —CH₂OH, —CH₂O-alkyl, or —COOH;

Ar₁ is an unsubstituted aromatic or heteroaromatic group or an aromatic or heteroaromatic group substituted with one or more substituent selected from the group consisting of (C₁₋₆)alkylamino, di-(C₁₋₆)-alkylamino, (C₁₋₆)alkoxy, carboxy, hydroxyl, cyano, halo, trifluoromethyl, nitro, amino, (C₁₋₆)acylamino, (C₁₋₆)alkylthio, (C₁₋₆)hydroxyalkyl, (C₁₋₆)alkylsulfonyl, and (C₁₋₆)haloalkyl;

Z₂ represents a group of the formula

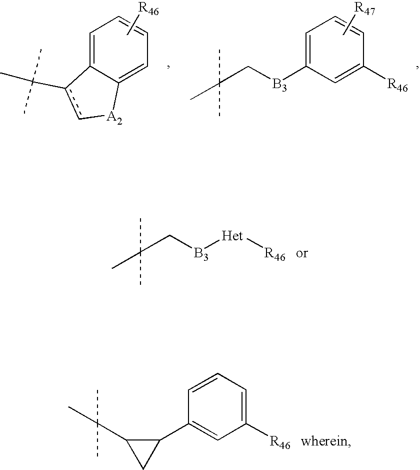

R₄₆ and R₄₇ are each independently hydrogen, halogen, (C₁₋₆)alkoxy, —OAr₁, (C₁₋₆)alkyl, —CF₃, COORᵢ, CONHRᵢ, —CN, —OH, CORᵢ, —S—(C₁₋₆)-alkyl, or —SO₂₋(C₁₋₆)-alkyl;

A₂ is CH₂, O, NH, NRᵢ, S, SO, SO₂, CH₂—CH₂, CH₂O, CHOH, or C(O), where Rᵢ is as defined above;

B₃ is CHRᵢ, C(Rᵢ)₂, (C₁₋₆)alkyl, C(O), —CHOH, —CH₂—O, —CH═CH, CH₂—C(O), CH₂—S, CH₂—S(O), CH₂—SO₂, —CHCO₂Rᵢ, or —CH—N(Rᵢ)₂, where Rᵢ is as defined above; and

Het is a heterocycle, such as furan, thiophene, or pyridine,

or an enantiomer, diastereoisomer, N-oxide, crystalline form, hydrate, solvate, pharmacologically active metabolite, prodrug, or pharmaceutically acceptable salt thereof.

Preferred molecules are:

3-[(3-bromobenzoyl)amino]-1-(1-indanyl)-3-methylpirrolidin-2-one,

3-[(3-chlorobenzoyl)amino]-1-(3-chlorophenethyl)-3-methylpyrrolidin-2-thione,

3-[(3-chlorobenzoyl)amino]-1-(1-indanyl)-3-methylpirrolidin-2-thione, and

3-[(6-chloropyridin-2-yl)carboxamido]-1-(1-indanyl)-3-methylpyrrolidin-2-thione

(5) Compounds with a structure represented by general formula V.

Ar₂—G₁—Ar₃ V-A

wherein

Ar₂ is a heteroaryl group,

Ar₃ is an aryl group, where

Ar₂ and Ar₃ are optionally substituted with one or more substituents selected from the group consisting of —F, —Cl, —Br, —I, —ORⱼ, —SRⱼ, —SORⱼ, —SO₂Rⱼ, —SO₂NRⱼRₖ, —OCORⱼ, —OCONRⱼRₖ, —NRCORₖ, —NRCO₂Rₖ, —CN, —NO₂, —CO₂Rⱼ, —CONRⱼRₖ, —C(O)Rⱼ, —CH(ORⱼ)Rₖ, —CH₂(ORⱼ), —Rⱼ, and —A—(CH₂)ₙ—NRⱼRₖ, wherein Rⱼ and Rₖ are selected independently from the group consisting of H, CF₃, (C₁₋₁₀)alkyl, cycloalkyl, alkyl-aryl, alkyl-heteroaryl, heterocycloalkyl, aryl, or Rⱼ and Rₖ may combine to form a C₁₋₅ methylene chain, and A is defined as CH₂, O, NH, S, SO, SO₂ and n is 1, 2, 3, or 4,

G₁ is selected from the group consisting of —NH—, —S, —O—, —CO—, —CONH—, —CONHCH₂—, —CH₂CONH—, —CH₂NHNH—, —CH₂NHNHCH₂—, —C═NO—CH₂—, —CH₂NHCH₂—, —CH₂CH₂NH—, —NHCH₂CO—, —NHCH₂CHOH—, —NHCH₂NHNH—, —NHCONH—, or G₁ is a cyclic group selected from the group consisting of cyclopentane, cyclopentadiene, furan, thiofuran, pyrrolidine, pyrrole, 2-imidazoline, 3-imidazoline, 4-imidazoline, imidazole, pyrazoline, pyrazolidine, imidazolidine, oxazole, 2-oxazole, thiazole, isoxazole, isothiazole, 1H-1,2,4-triazole, 1H-1,2,3-triazole, 1,2,4-oxathiazole, 1,3,4-oxathiazole, 1,4,2-dioxazole, 1,4,2-oxathiazole, 1,2,4-oxadiazole, 1,2,4-thiadiazole, 1,2,5-oxadiazole, 1,2,5-thiadiazole, 1,3,4-oxadiazole, 1,3,4-thiadiazole, 1H-tetrazole, cyclohexane, piperidine, tetrahydropyridine, 1,4-dihydropyridine, pyridine, benzene, tetrahydropyran, 3,4-dihydro-2H-pyran, 2H-pyran, 4H-pyran, tetrahydrothiopyran, 3,4-dihydro-2H-thiopyran, 2H-thiin, 4H-thiopyran, morpholine, thiomorpholine, piperazine, pyridazine, pyrimidine, pyrazine, 1,2,4-triazine, 1,2,3-triazine, 1,3,5-triazine, and 1,2,4,5-tetrazine groups,

or an enantiomer, diastereoisomer, N-oxide, crystalline form, hydrate, solvate, pharmacologically active metabolite, prodrug, or pharmaceutically acceptable salt thereof.

Preferred groups that Ar₃ represents independently are phenyl, benzyl, naphthyl, fluorenyl, anthrenyl, indenyl, phenanthrenyl, and benzonaphthenyl groups,

Preferred groups that Ar₂ represents independently are thiazolyl, furyl, pyranyl, 2H-pyrrolyl, thienyl, pyrrolyl, imidazolyl, pyrazolyl, pyridyl, pyrazinyl, pyrimidinyl, pyridazinyl, benzothiazolyl, benzimidazolyl, 3H-indolyl, indolyl, indazolyl, purinyl quinolizinyl, isoquinolyl, quinolyl, phthalizinyl, naphthyridinyl, quinazolinyl, cinnolinyl, isothiazolyl, quinoxalinyl, indolizinyl, isoindolyl, benzothienyl, benzofuranyl, isobenzofuranyl, and chromenyl.

Further preferred is where together Ar₃ is selected from the group consisting of phenyl, benzyl, naphthyl, fluorenyl, anthrenyl, indenyl, phenanthrenyl and benzonaphthenyl groups and Ar₂ is selected from the group consisting of thiazolyl, furyl, pyranyl, 2H-pyrrolyl, thienyl, pyrrolyl, imidazolyl, pyrazolyl, pyridyl, pyrazinyl, pyrimidinyl, pyridazinyl, benzothiazolyl, benzimidazolyl, 3H-indolyl, indolyl, indazolyl, purinyl, quinolizinyl, isoquinolyl, quinolyl, phthalizinyl, naphthyridinyl, quinazolinyl, cinnolinyl, isothiazolyl, quinoxalinyl, indolizinyl, isoindolyl, benzothienyl, benzofuranyl, isobenzofuranyl and chromenyl groups.

In yet another embodiment; compounds of the present invention can be represented by formula V-B:

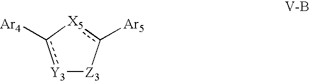

wherein

X₅, Y₃, and Z₃ are independently selected from the group consisting of N, O, S, C, and CO wherein at least one of X₅, Y₃, and Z₃ is a heteroatom;

Ar₄ and Ar₅ are independently selected from the group consisting of a heterocyclic or fused heterocyclic group containing 1 to 4 heteroatoms selected from the group consisting of N, O, and S and an aromatic group selected from the group consisting of phenyl, benzyl, 1-naphthyl, 2-naphthyl, fluorenyl, anthrenyl, indenyl, phenanthrenyl, and benzonaphthenyl, wherein Ar₄ and Ar₅ are optionally substituted with one or more substituents selected from the group consisting of —F, —Cl, —Br, —I, —ORⱼ, —SRⱼ, —SORⱼ, —SO₂Rⱼ, —SO₂NRⱼRₖ, —OCORⱼ, —OCONRⱼRₖ, —NRCORₖ, —NRCO₂Rₖ, —CN, —NO₂, —CO₂Rⱼ, —CONRⱼRₖ, —C(O)Rⱼ, —CH(ORⱼ)Rₖ, —CH₂(ORⱼ)—Rⱼ, and —A—(CH₂)ₙ—NRⱼRₖ; wherein Rⱼ and Rₖ are selected independently from the group consisting of H, CF₃, (C₁₋₁₀)alkyl, cycloalkyl, alkyl-aryl, alkyl-heteroaryl, heterocycloalkyl, aryl, or Rⱼ and Rₖ may combine to form a C₁₋₅ methylene chain, A is defined as CH₂, O, NH, S, SO, SO₂,

and n is 1,2,3, or 4,

or an enantiomer, diastereoisomer, N-oxide, crystalline form, hydrate, solvate, pharmacologically active metabolite, prodrug, or pharmaceutically acceptable salt thereof.

The heterocyclic or fused heterocylic group preferably is a member selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl.

In a preferred embodiment of the invention, the compound is a member selected from the group consisting of 3-(2-pyridyl)-5-(3,5-dichlorophenyl)-1,2,4-oxadiazole, 3-(2-pyridyl)-5-(3-methoxyphenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(2,3-difluorophenyl)-1,2,4-oxadiazole, 3-(5-methoxypyrid-2-yl)-5-(3-cyanophenyl)-1,2,4-oxadiazole, and 3-(2-quinolinyl)-5-(3-cyanophenyl)-1,2,4-oxadiazole.

In another embodiment of the invention, the compound is a member selected from the group consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(3-cyanophenyl)-4-(5-methoxypyrid-2-yl)-1,3-oxazole, 2-(3-cyanophenyl)-4-(2-quinolyl)-1,3-oxazole, 2-[3-chlorophenyl]-4-[pyridin-2-yl]-1,3-oxazole, 2-(2,5,6-trifluorophenyl)-4-(2-pyridyl)-1,3-oxazole, and 2-(3-nitrophenyl)-4-(2-pyridyl)-1,3-oxazole.

The present invention also includes the enantiomers, diastereomers, N-oxides, crystalline forms, hydrates, solvates and pharmaceutically acceptable salts of the compounds of general formulas I, I-A, II-A, II-B, II-C, III, IV, V-A, and V-B (hereinafter referred to collectively as “formulas I-V”) that are selective antagonists of mGlu5 receptors.

The present invention also includes metabolites of the compounds of formulas I-V that are selective mGlu5 antagonists, hereinafter referred to as active metabolites.

The present invention also contemplates prodrugs which are metabolized in the body to generate the compounds of formulas I-V that are selective mGlu5 antagonists.

In another embodiment, the present invention provides pharmaceutical compositions comprising a selective mGlu5 antagonist, e.g., a compound of formulas I-V that is a selective mGlu5 antagonist and enantiomers, diastereomers, N-oxides, crystalline forms, hydrates, solvates or pharmaceutically acceptable salts thereof, in admixture with a pharmaceutically acceptable diluent or carrier such as those disclosed.

In another embodiment, the present invention provides the use of at least one compound that is a selective mGlu5 antagonist, e.g., at least one compound of one of formulas I-V that is a selective mGlu5 antagonist, in an amount effective for reducing the frequency of bladder contractions due to bladder distension by administering it to a mammal, including a human, in need of such treatment.

In yet another embodiment, the invention provides the use of at least one compound that is a selective mGlu5 antagonist, e.g., at least one compound of one of formulas I-V that is a selective mGlu5 antagonist, in an amount effective for increasing urinary bladder capacity and the time-interval between one micturition and the following by administering it to a mammal, including a human, in need of such treatment.

In another embodiment, the invention provides the use of at least one compound that is a selective mGlu5 antagonist, e.g., at least one compound of one of formulas I-V that is a selective mGlu5 antagonist, in an amount effective for treating neuromuscular dysfunction of the urinary tract in a patient in need of such treatment to ameliorate at least one condition selected from the group consisting of urinary urgency, overactive bladder, increased urinary frequency, decreased urinary compliance (decreased bladder storage capacity), cystitis (including interstitial cystitis), incontinence, urine leakage, enuresis, dysuria, urinary hesitancy and difficulty in emptying the bladder.

A person of ordinary skill in the art will appreciate that the aforementioned compounds may contain one or more chiral centers, and thus can exist as racemic mixtures. For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that invention compounds may exist in polymorphic forms wherein a compound is capable of crystallizing in different forms. Suitable methods for identifying and separating polymorphisms are known in the art.

In yet another embodiment, the invention provides a method for identifying a compound useful for treating neuromuscular dysfunction of the lower urinary tract comprising:

(a) individually measuring the binding affinity of a test compound for the mGlu5 receptor, mglul receptor and Group II mGlu receptors;

(b) identifying those test compounds that:

(1) bind to a mGlu5 receptor with an affinity of at least 10⁻⁶ M, and

(2) bind to a mGlu5 receptor with an affinity at least 10-fold stronger than the affinity for a mGlu1 receptor and Group II mGlu receptors.

In certain embodiments, the method for identifying a compound useful for treating neuromuscular dysfunction of the lower urinary tract further comprises:

(c) individually determining the ability of each of the compounds identified in steps (a) and (b) above to act as an antagonist or inverse agonist at the mGlu5 receptor.

In yet another embodiment, the invention provides a method for identifying a compound useful for treating neuromuscular dysfunction of the lower urinary tract comprising:

(a) individually measuring the binding affinity of a test compound for the mGlu5 receptor, mGlu1 receptor, Group II and Group III mGlu receptors;

(b) identifying those test compounds that:

(1) bind to a mGlu5 receptor with an affinity of at least 10⁻⁶ M, and

(2) bind to a mGlu5 receptor with an affinity at least 1 0-fold stronger than the affinity for the mGlu1 receptor, Group II and Group III mGlu receptors and

(c) individually measuring the ability of each of the compounds identified in step (b) to act as an antagonist or inverse agonist at the mGlu5 receptor.

Preferably, the activity of compounds identified in steps (a), (b), and (c) of the methods described above is confirmed by evaluating the activity of the compound in treatment of lower urinary tract disease in humans or an animal model system. More preferably the compounds identified exhibit activity in at least one of the following biological parameters:

(1) inhibition of volume-induced rhythmic bladder voiding contractions in anesthetized rats;

(2) increase in bladder volume capacity in conscious rats.

In certain embodiments, the invention is also related to compounds identified by the aforementioned methods.

In certain embodiments a selective mGlu5 antagonist is used to treat the aforementioned disorders by administering the antagonist in combination with known antimuscarinic drugs. Analogously, a selective mGlu5 antagonist may be administered in combination with α1-adrenergic antagonists, for the therapy of lower urinary tract symptoms, whether or not these are associated with BPH.

In a similar fashion a selective mGlu5 antagonist may be administered in combination with one or more antagonists of the 5-HT_{1A} receptor.

In a similar fashion a selective mGlu5 antagonists may be administered in combination with one ore more inhibitors of the enzyme cyclooxygenase (COX), which may be selective or non-selective for the COX2 isozyme, and derivatives thereof, such as NO releasing COX ester or amide inhibitors.

### BRIEF DESCRIPTION OF DRAWINGS

FIG. 1 shows the change over time of bladder volume capacity (BVC) in rats after intravenous administration of vehicle (circles), 3.0 and 10 mg/kg of the compound 2-methyl-6-(phenylethynyl)pyridine (MPEP) or 50 mg/kg of 2-methyl-6-(2-phenylethenyl)pyridine (SIB 1893) (squares). Data represent the percentage change relative to basal values at different times after treatment. n=number of rats/group.

FIG. 2 shows the change over time of micturition pressure (MP) in rats after intravenous administration of vehicle (circles), 3.0 and 10 mg/kg of the compound 2-methyl-6-(phenylethynyl)pyridine (MPEP) or 50 mg/kg of 2-methyl-6-(2-phenylethenyl) pyridine (SIB 1893) (squares). Data represent the percentage changes relative to basal values at different times after treatment. n=number of rats/group.

FIG. 3 shows the change over time of BVC in rats after oral administration of vehicle (circles), or 3.0, 10 or 30 mg/kg of the compound 2-methyl-6-(phenylethynyl)pyridine (MPEP) (squares). Data represent the percentage changes relative to basal values at different times after treatment. n=number of rats/group.

FIG. 4 shows the change over time of MP in rats after oral administration of vehicle (circles), or 3.0, 10 or 30 mg/kg of the compound 2-methyl-6-(phenylethynyl)pyridine (MPEP) (squares). Data represent the percentage changes relative to basal values at different times after treatment. n=number of rats/group.

FIG. 5 shows the change over time of BVC in rats with bladder infused with dilute acetic acid after oral administration of vehicle (circles), 10 mg/kg of the compound 2-methyl-6-(phenylethynyl)pyridine (MPEP) (squares; left panel), or 1 mg/kg of indomethacin (squares; right panel). Data represent the percentage changes relative to basal values at different times after treatment. n=number of rats/group.

FIG. 6 shows the change over time of MP in rats with bladder infused with dilute acetic acid after oral administration of vehicle (circles), 10 mg/kg of the compound 2-methyl-6-(phenylethynyl)pyridine (MPEP) (squares; left panel), or 1 mg/kg of indomethacin (squares; right panel). Data represent the percentage changes relative to basal values at different times after treatment. n=number of rats/group.

### DETAILED DESCRIPTION OF THE INVENTION

All cited patents, patent applications and literature references are hereby incorporated herein by reference in their entireties. In the case of inconsistencies, the present disclosure, including definitions, will prevail unless the context requires otherwise.

The present invention is related to methods of using antagonists and/or inverse agonists of the mGlu5 receptor for treating neuromuscular dysfunction of the lower urinary tract. The mGlu5 receptor is preferably human mGlu5.

The invention is based on our findings that:

i) a compound fimctionally acting as a selective antagonist and/or inverse agonist of the metabotropic glutamate receptor, mGlu5 receptor, can inhibit the micturition reflex inducing a dose-dependent long-lasting block of bladder contractility when bladder is filled to the threshold volume, without affecting the amplitude of bladder contraction when the effect is ceased;

ii) a compound functionally acting as a selective antagonist and/or inverse agonist of the metabotropic glutamate receptor, mGlu5 receptor, can increase the bladder volume capacity as measured in cystometric recordings at doses that do not impair bladder contractility; and

iii) a compound functionally acting as a selective antagonist and/or inverse agonist of the metabotropic glutamate receptor, mGlu5 receptor, can increase the time interval between one micturition and the following, as measured in cystometric recordings.

Compounds of general formula I may be synthesized as described in publication WO 99/02497.

Synthesis of compounds represented by formula I-A is described in publication WO 02/062323.

Compounds of general formulas II-A, II-B, and II-C may be synthesized as described in publication WO 02/46166.

Compounds of general formula III may be synthesized as described in publication WO 01/16121.

Compounds of general formula IV may be synthesized as described in publication WO 00/69816.

Compounds of general formulas V-A and V-B may be synthesized as described in publication WO 01/12627.

The compounds disclosed in the aforementioned publications are incorporated herein by reference.

The disclosures of U.S. patent publications 2002/0128263 A1, 2002/0188128 A1, and 2003/0022907 A1 are incorporated herein by reference. In certain embodiments, the compounds disclosed in the aforementioned U.S. patent publications may be excluded from the claims.

Pharmacological blocking of the mGlu5 receptor leads to positive effects in the management of neuromuscular dysfunction of the lower urinary tract.

The antagonists and/or inverse agonists of the present invention are related to compounds of formulas I-V as disclosed above, including the enantiomers, diastereomers, N-oxides, crystalline forms, hydrates, solvates or pharmaceutically acceptable salts of these compounds, as well as active metabolites of these compounds having the same type of activity. Preferably, the compounds of formulas I-V are selective mGlu5 antagonists.

An antagonist of mGlu5 receptor is a substance which diminishes or abolishes the effect of a ligand (agonist) which typically activates mGlu5 receptor. The antagonist may be, for example, a chemical antagonist, a pharmacokinetic antagonist, an antagonist by receptor block, a non-competitive antagonist or a physiological antagonist.

A chemical antagonist is a substance wherein the antagonist binds the ligand in solution so the effect of the ligand is lost. A pharmacokinetic antagonist is one which effectively reduces the concentration of the active drug at its site of action, for example, by increasing the rate of metabolic degradation of the active ligand. Antagonism by receptor-block involves two important mechanisms: reversible competitive antagonism and irreversible, or non-equilibrium competitive antagonism. Reversible competitive antagonism occurs when the rate of dissociation of the antagonist molecules is sufficiently high such that, on addition of the ligand, displacement of chemical antagonist molecules from the receptors effectively occurs. Of course the ligand cannot evict a bound antagonist molecule, or vice versa. Irreversible or non-equilibrium competitive antagonism occurs when the antagonist dissociates very slowly, or not at all, from the receptor with the result that no change in the antagonist occupancy takes place when the ligand is applied. Thus, the antagonism is insurmountable. Non-competitive antagonism describes the situation where the antagonist blocks at some point in the signal transduction pathway leading to the production of a response by the ligand.

Physiological antagonism is a term used loosely to describe the interaction of two substances whose opposing actions in the body tend to cancel each other out. An antagonist can also be a substance which diminishes or abolishes expression of functional mGlu5 receptor. Thus an antagonist can be, for example, a substance which diminishes or abolishes expression of the gene encoding mGlu5 receptor, diminishes or abolishes translation of mGlu5 receptor RNA, diminishes or abolishes post-translational modification of mGlu5 receptor protein or diminishes or abolishes the insertion of mGlu receptor into the cell membrane.

An inverse agonist of mGlu5 receptor is a substance which preferentially binds to the inactive state of the receptor (in contrast to the agonists that bind preferentially to the active state of the receptor), and therefore avoids the stimulation of the receptor by the agonist.

In general, the in vivo activity of inverse agonists is similar to that of antagonists and for the sake of clarity inverse agonists will be defined as antagonists in the present application.

An antagonist for use in the invention may be a relatively non-specific antagonist which is an antagonist of mGlu5 receptors in general. Preferably, however, an antagonist selectively antagonizes Group I mGlu receptors. More preferably, an antagonist used in the invention is a selective mGlu5 receptor antagonist. A selective mGlu5 receptor anagonist is one which antagonizes mGlu5 receptor, but antagonizes other mGlu receptors only weakly or substantially not at all. Most preferred antagonists are those that selectively antagonize mGlu5 receptor at low concentration, for example those that cause a level of antagonism of 50% or greater at a concentration of 1000 nM or less. Selective mGlu5 antagonists may thus exhibit, e.g., at least about a 10-fold, at least about a 20-fold, at least about a 50-fold, at least about a 100-fold, at least about a 250-fold, at least about a 500-fold, or at least about a 1000-fold greater activity at an mGlu5 receptor than at an mGlu1 receptor.

Accordingly, selective mGlu5 antagonist have the following properties.

(1) Significant mGlu5 antagonist activity: Useful compounds preferably exhibit antagonist potency (measured as IC₅₀ or Ki) between about 1000 and 0.1 nM. Without limiting the present disclosure, as described in more detail below, potency may be measured by determining the antagonist activity of compounds in vivo or in vitro, including cell extracts or fractions of extracts. Inhibitory potency may also be determined using, as non-limiting examples, native or recombinant mGlu5 receptors, enzymes that are expressed constitutively or that have been induced, and enzymes that have expressed in native or non-native species and/or cell types (W. Sporen , Trends in Pharmacol. Sci. 23:331-337, 2001).

(2) Selectivity: Preferred compounds exhibit at least about 10-fold greater antagonist potency for mGlu5 receptors, compared to each of mGlu1 receptors and Group II mGlu receptors. More preferred are compounds that exhibit at least about a 20-fold, at least about a 50-fold, at least about a 100-fold, at least about a 250-fold, at least about a 500-fold, or at least about a 1000-fold greater antagonist potency for mGlu5 receptors, compared to each of mGlu1 receptors and Group II mGlu receptors. Accordingly, compounds belonging to this general class are suitable candidates for testing according to the methods taught below. Selectivity may be measured as the ratio of Kᵢ or IC₅₀ for different receptors.

Screening candidate compounds to identify those compounds that are useful in practicing the present invention including, for example and without limitation, involves:

1) evaluating their antagonist potency and selectivity for mGlu5 receptors, compared to one or more mGlu1 receptor or Group II mGlu receptor and optionally a Group III mGlu receptor, preferably to at least one receptor from each of the mGlu1 and Group II mGlu groups of receptors and optionally a Group III mGlu receptor; and

2) confirming their pharmacological activity using one or more animal model systems for neuromuscular dysfunction of the lower urinary tract.

Commonly used in vitro assays for assessing antagonist activity at metabotropic glutamate receptors are found, for example, in Carroll et al. (Mol Pharmacol 59 : 965-973, 2001). In preferred embodiment, measurement of antagonist activity at metabotropic glutamate receptors is performed using one or more of the assays described in the examples set forth below. Using one or more of said assays, the antagonist activity at metabotropic glutamate receptors of a test compound can be measured for different isozymes, and the concentration inhibiting binding by 50% (IC₅₀) can be calculated using regression analysis, or equivalent computational methods that are well-known in the art (Tallarida et al., Manual of Pharmacologic Calculations. Springer-Verlag, pp. 10-12, 1981).

A compound is considered to be a “selective” mGlu5 antagonist if it exhibits a selectivity ratio of at least 10-fold, i.e., the IC₅₀ or Ki for mGlu5 is at least 10-fold below the IC₅₀ or Ki for one or more mGlu1 receptor or Group II mGlu receptor or, preferably to at least one receptor from each of the mGlu1 and Group II mGlu receptors. More preferably a selective mGlu5 antagonist as described herein further exhibits a selectivity ratio of at least 10-fold for one or more Group III mGlu receptor.

Once a compound is identified as a selective mGlu5 antagonist, its pharmacological activity can be confirmed using one or more animal model systems for neuromuscular dysfunction of the lower urinary tract.

A useful animal model system for measuring such pharmacological activity is, without limitation, volume-induced rhythmic bladder voiding contractions in anesthetized rats. In this method, the urinary bladder is catheterized through the external urethra with a polyethylene tubing filled with physiological saline. The external urethra is then ligated and connected to a pressure recording device. The bladder is then filled with saline until reflex voiding contractions occur, after which the frequency of the voiding contractions is measured for 15 min. Test compounds are then administered intravenously and their effect evaluated for the following 60 min. This method is described in more detail in Example 3 below, and was originally used to validate the predictive qualities of selective mGlu5 antagonists for urinary tract disorders. This model has been validated by the use of different reference standards (Guarneri et al., Pharmacol. Res. 27:173-187, 1993).

Other animal models useful to assess activity of the selective mGlu5 antagonists on the lower urinary tract are given in Examples 4-5 of the current application. They are based on cystometric recording of bladder activity in conscious rats instrumented in order to measure bladder pressure during constant infusion of the bladder with saline or very diluted acetic acid. These methods are widely used and accepted by researchers skilled in this field and foresee a period of infusion of about five hours after administration of test compounds with continuous monitoring of bladder performance and assessment of intervals between micturitions and peak micturition pressure.

A “metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9^{th} Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

A “prodrug” of a compound disclosed herein is an inactive substance that converts into an active form of the disclosed compounds in vivo when administered to a mammal.

In certain embodiments lower urinary tract disease is treated by administering a selective mGlu5 antagonist in combination with an antagonist of one or more additional class of receptors. In preferred embodiments a selective mGlu5 antagonist is administered in combination with an antagonist of an α1-adrenergic, 5-HT_{1A} or muscarinic receptor.

In further embodiments, lower urinary tract disease is treated by administering a selective mGlu5 antagonist in combination with one or more inhibitor of the cyclooxygenase enzyme, which may inhibit both COX1 and COX2 isozymes or which may, alternatively, be selective for COX2 isozyme, and NO donor derivatives thereof.

Examples of antimuscarinic drugs for administration in combination with a selective mGlu5 antagonist are oxybutynin, tolterodine, darifenacin, and temiverine.

A selective mGlu5 antagonist may be administered in combination with α1-adrenergic antagonists, for the therapy of lower urinary tract symptoms, whether or not these are associated with BPH. Preferred α1-adrenergic antagonists suitable for administration in combination with a selective mGlu5 antagonist are, for example, prazosin, doxazosin, terazosin, alfuzosin, and tamsulosin. Additional al-adrenergic antagonists suitable for administration in combination with a selective mGlu5 antagonist are described in U.S. Pat. Nos. 5,990,114; 6,306,861; 6,365,591; 6,387,909; and 6,403,594.

Examples of 5-HT_{1A} antagonists that may be administered in combination with a selective mGlu5 antagonist are found in Leonardi et al., J. Pharmacol. Exp. Ther. 299: 1027-1037, 2001 (e.g., Rec 15/3079), U.S. Pat. No. 6,071,920, other phenylpiperazine derivatives described in WO 99/06383 and pending U.S. patent applications Ser. Nos. 10/266,088 and 10/266,104 filed on Oct. 7, 2002. Additional 5-HT_{1A} antagonists include DU-125530 and related compounds described in U.S. Pat. No. 5,462,942 and robalzotan and related compounds described in WO 95/11891. Each of the foregoing are non-limiting examples of 5-HT_{1A} antagonists that may be administered in combination with a selective mGlu5 antagonists.

Examples of selective COX2 inhibitors that may be administered in combination with selective antagonists of the mGlu5 receptor are, without limitation, nimesulide, meloxicam, rofecoxib, celecoxib, parecoxib and valdecoxib. Additional examples of selective COX2 inhibitors are described, without limitation, in U.S. Pat. No. 6,440,963. Examples of non-selective COX1-COX2 inhibitors are, without limitation, acetylsalicylic acid, niflumic acid, flufenamic acid, enfenamic acid, meclofenamic acid, tolfenamic acid, thiaprophenic acid, ibuprofen, naproxen, ketoprofen, flurbiprofen, furprofen, indomethacin, acemethacin, proglumethacin, ketorolac, diclofenac, etodolac, sulindac, fentiazac, tenoxicam, lomoxicam, cynnoxicam, ibuproxam, nabumetone, tolmetin, amtolmetin. Accordingly, each of the foregoing are non-limiting examples of COX inhibitors that may be administered in combination with a selective mGlu5 antagonists.

Examples of derivatives of COX inhibitors that may be administered in combination with selective mGlu5 antagonists are derivatives of COX inhibitors bearing nitrate (nitrooxy) or nitrite groups, such as those given, for example, in WO 98/09948, able to release NO in vivo.

### Chemical Definitions

The following definitions of general terms used in the present descriptions of formulas I-V apply irrespective of whether the terms in question appear alone or in combination.

The terms “hydroxy” and “hydroxyl” are synonymous and refer to a group —OH.

The term “oxo” and “keto” are synonymous and refer to a group ═O.

The term “carbonyl” refers to a group —C(═O)—.

The term “alkylcarbonyl” refers to a group —C(═O)-alkyl.

The term “alkylthio” refers to a group —S-alkyl.

The term “sulfonyl” refers to a group —SO₂—.

The term “nitro” refers to a group —NO₂.

The term “amino” refers to a group —NH₂.

The term “cyano” refers to a group —C≡N.

The term “aralkyl” refers to a group -alkyl-aryl.

The term “alkanoyloxy” refers to a group —O-alkanoyl.

The term “acyl”, whether used alone, or within a term such as “acylamino”, denotes a radical provided by the residue after removal of hydroxyl from an organic acid.

The term “acylamino” denotes an amino radical substituted with an acyl group. Examples of an “acylamino” radical are acetylamino or acetamido (CH₃C(═O)—NH—) where the amine may be further substituted with alkyl, aryl or aralkyl.

The term “alkoxy” denotes linear or branched oxy-containing radicals each having alkyl portions of one to about ten carbon atoms, such as methoxy radical. “Lower alkoxy” denotes a lower alkyl group which is bound via an oxygen atom. Examples of such lower alkoxy groups include methoxy, ethoxy, n-propoxy, isopropoxy, n-butoxy, tert-butoxy and the like.

The term “alkoxycarbonyl” denotes a radical containing an alkoxy radical, as defined above, attached via an oxygen atom to a carbonyl radical, i.e., an ester radical. Preferably, “lower alkoxycarbonyl” denotes alkoxy radicals having one to five carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “alkyl” denotes saturated straight or branched chain hydrocarbon radicals having in the range of about one to twelve carbon atoms. Examples of alkyl include, without limitation, methyl, ethyl, n-propyl, isopropyl, n-butyl, sec-butyl, tert-butyl, pentyl, isopentyl, neopentyl, hexyl, isohexyl, heptyl, isoheptyl, and the like. “Lower alkyl” denotes straight-chain or branched saturated hydrocarbon residues with one to six carbon atoms, preferably with one to four carbon atoms. The term “substituted alkyl” denotes alkyl radicals wherein at least one hydrogen is replaced by one more substituents such as, for example, hydroxy, alkoxy, mercapto, aryl, heterocycle, halogen, trifluoromethyl, pentafluoroethyl, cyano, cyanomethyl, nitro, amino, amide, amidine, amido, carboxyl, carboxamide, carbamate, ester, sulfonyl, sulfonamide, and the like.

The term “alkenyl” refers to linear or branched radicals of two to about twelve carbon atoms having at least one carbon-carbon double bond. Preferred alkenyl radicals are “lower alkenyl” radicals having about two to 12 carbon atoms, more preferred with about two to six carbon atoms. Examples of such radicals include ethenyl, n-propenyl, butenyl, and the like. The term “substituted alkenyl” refers to an alkenyl wherein at least one hydrogen is replaced by one or more substituents or groups independently selected for each position.

The term “cycloalkenyl” refers to ring-containing alkenyl radicals having at least one carbon-carbon double bond in the ring, and having in the range of about three up to twenty carbon atoms, and “substituted cycloalkenyl” refers to cyclic alkenyl radicals further bearing one or more substituents as set forth above.

The term “alkenylene” refers to straight or branched chain divalent alkenyl groups having at least one carbon-carbon double bond, and having in the range of about two up to twelve carbon atoms (with divalent alkenyl groups having in the range of about two up to six carbon atoms), and “substituted lower alkenylene” refers to divalent alkenyl radicals further bearing one or more substituents as set forth above.

The term “alkynyl” refers to linear or branched radicals of two to about twelve carbon atoms having at least one carbon-carbon triple bond. Preferred alkynyl radicals are “lower alkynyl” radicals having two to about six carbon atoms. Examples of such radicals include, without limitation, ethynyl, n-propynyl, butynyl, and the like. The term “substituted alkynyl” refers to an alkynyl wherein at least one hydrogen is replaced by one or more substituents or groups independently selected for each position as forth above.

The term “cycloalkynyl” refers to ring-containing alkynyl radicals having at least one carbon-carbon triple bond in the ring, and having in the range of about three up to twenty carbon atoms, and “substituted cycloalkynyl” refers to cyclic alkynyl radicals further bearing one or more substituents as set forth above.

The term “alkynylene” refers to straight or branched chain divalent alkynyl groups having at least one carbon-carbon triple bond, and having in the range of about two up to twelve carbon atoms (preferably with divalent alkynyl groups having about two carbon atoms), and “substituted alkynylene” refers to divalent alkynyl radicals further bearing one or more substituents as set forth above.

The term “lower alkylamino” refers to an amino group in which the nitrogen atom of the amino (as defined above) is once substituted with an alkyl (as defined above). Preferably, the alkyl radical is one to five carbon atoms in length. The alkyl radical of an alkylamino may be substituted. Preferred substituents for the alkyl radical of an alkylamino group are, for example, hydroxyl, alkoxyl, arylalkoxyl, amino, acylamino and cyanoamino groups. Therefore, the term “substituted alkylamino” refers to an alkyl radical attached to an amino group wherein at least one hydrogen of the alkyl radical is replaced by one or more substituents independently selected for each position as forth above.

The term “alkanoyl” refers to a straight or branched alkyl chain attached to a carbonyl group. The alkanoyl radicals may be substituted or unsubstituted. Examples of “alkanoyl” groups include, without limitation, formyl, acetyl (ethanoyl), propanoyl, isopropanoyl, butanoyl, isobutanoyl, valeryl (pentanoyl), isovaleryl, pivaloyl, hexanoyl, t-butanoyl, pentanoyl, 3-methylpentanoyl or the like. Preferred alkanoyl radicals are “lower alkanoyl” radicals having about one to five carbon atoms, such as formyl, acetyl or propanoyl.

The terms “carboxyalkyl” refers to radicals having a carboxy radical as defined attached to an alkyl radical.

The terms “halo” or “halogen” refer to fluoride, chloride, bromide or iodide atoms. The term “haloalkyl” denotes radicals wherein any one or more of the alkyl carbon atoms is substituted with halo as defined above. Specifically embraced are monohaloalkyl, dihaloalkyl and polyhaloalkyl radicals. A monohaloalkyl radical, for example, may have either an iodo, bromo, chloro or fluoro atom within the radical. Dihalo and polyhaloalkyl radicals may have two or more of the same halo atoms or a combination of different halo atoms.

The term “lower haloalkyl” denotes radicals having one to six carbon atoms wherein one or more hydrogen atoms has been replaced with a halogen atom. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” denotes linear or branched alkyl radicals having one to about ten carbon atoms any one of which may be substituted with one or more hydroxyl radicals. More preferred hydroxyalkyl radicals are “lower hydroxyalkyl” radicals having one to five carbon atoms and one or more hydroxyl radicals. Examples of such radicals include hydroxymethyl, hydroxyethyl, hydroxypropyl, hydroxybutyl and hydroxyhexyl.

The term “arylamino” denotes amino groups which have been substituted with one or two aryl radicals, such as N-phenylamino.

The terms “carboxy” or “carboxyl”, whether used alone or with other terms, such as “carboxyalkyl”, denote —CO₂H.

The term “aryloxy” denotes aryl radicals, as defined above, attached to an oxygen atom. Examples of such radicals include phenoxy.

The term “dialkylamino” denotes an amino group in which the N atom of the amino (as defined above) is twice substituted with alkyl (as defined above) radicals. Preferably, the alkyl radicals are independently one to six carbon atoms in length. One or both alkyl radicals of an alkylamino may be substituted. Preferred independent substituents for the alkyl radicals of dialkylamino group are, for example, hydroxyl, alkoxyl, arylalkoxyl, amino, acylamino and cyanoamino groups. Therefore, the term “substituted dialkylamino” refers to two alkyl radicals attached to an amino group wherein at least one hydrogen of one or both alkyl radicals is replaced by one or more substituents independently selected for each position.

The terms “heterocycle” and “heterocyclic” are synonymous and refer to ring-containing radicals having one or more heteroatoms (e.g., N, O, S) as part of the ring structure, and having in the range of three up to twenty atoms in the ring. Heterocyclic groups may be saturated, partially unsaturated containing one or more double bonds or fully unsaturated. Fully unsaturated heterocyclic group is synonymous with heteroaryl group. Heterocyclic groups may comprise one, two or three rings. When a heterocyclic groups comprises two or three rings, the rings may be attached in any configuration, e.g., in a fused or spiro configuration. Heterocyclic groups include, for example and without limitation, monocyclic heterocylic groups, containing one to four nitrogen atoms (e.g., pyrrolidinyl, imidazolidinyl, piperidino, piperazinyl, etc.); saturated three to six-membered heteromonocyclic group containing one to two oxygen atoms and one to three nitrogen atoms (e.g., morpholinyl, etc.) and bicyclic heterocyclic groups, e.g., azabicycloalkanyl and oxabicycloalkyl groups; unsaturated three to six-membered heteromonocyclic group containing one to two sulfur atoms and one to three nitrogen atoms (e.g., thiazolyl, etc.), imidazolyl, pyrimidinyl, isothiazolyl and isoxazolyl groups. The aforementioned are non-limiting examples. Additional examples of “heterocyclic” groups are set forth herein. The term “substituted heterocycle” refers to heterocycles further bearing one or more substituents as set forth above.

The term “aryl”, alone or in combination, means a carbocyclic aromatic system containing one, two or three rings wherein such rings may be attached together in a pendent manner or may be fused. The term “aryl” embraces aromatic radicals such as, for example and without limitation, phenyl, naphthyl, tetrahydronaphthyl, indane and biphenyl.

The term “heteroaryl” denotes fully unsaturated heterocyclic radicals. Examples of unsaturated heterocyclic radicals, also termed “heteroaryl” radicals include unsaturated five to six membered heteromonocyclic group containing one to four nitrogen atoms, such as furyl, pyrrolyl, thienyl, 1H-imidazolyl, 2H-imidazolyl, 4H-imidazolyl, 1H-pyrazolyl, 3H-pyrazolyl, 4H-pyrazolyl, 1,2-oxazolyl, 1,3-oxazolyl, 1H-[1,2,4]triazolyl, 4H-[1,2,4]triazolyl, 1H-[1,2,3]triazolyl, 2H-[1,2,3]triazolyl, 4H-[1,2,3]triazolyl, [1,2,4]oxadiazolyl, [1,3,4]oxadiazolyl, [1,2,3]oxadiazolyl, 1H-tetrazolyl, 2H-tetrazolyl, [1,2,3,4]oxatriazolyl, [1,2,3,5]oxatriazolyl, 1,3-thiazolyl, 1,2-thiazolyl, 1H-pentazolyl, pyridyl, pyrazinyl, pyrimidinyl, pyridazinyl, indolyl, quinolinyl and their dihydro derivatives. The term also denotes radicals where heterocyclic radicals are fused with aryl radicals. Examples of such fused bicyclic radicals include benzofuran, benzothiophene, and the like.

The term “cycloalkyl” denotes saturated hydrocarbon radicals having about three to twenty carbon atoms. More preferred cycloalkyl radicals are “lower cycloalkyl” radicals having three to seven carbon atoms. Examples include radicals such as cyclopropyl, cyclobutyl, cyclopentyl, cyclohexyl and cycloheptyl. “Substituted cycloalkyl” denotes cycloalkyl radicals further bearing one or more substituents as set forth herein.

The term “hydrocarbyl” refers to straight or branched chain univalent and bivalent radicals derived from saturated or unsaturated groups containing only carbon and hydrogen atoms, and having in the range of about 1 up to 12 carbon atoms. Exemplary hydrocarbyl groups include alkyl groups, alkenyl groups, dialkenyl groups, trialkenyl groups, alkynyl groups, alkadiynyl groups, alkatriynyl groups, alkenyne groups, alkadienyne groups, alkenediyne groups, and the like. The term “substituted hydrocarbyl” refers to hydrocarbyl groups further bearing one or more substituents.

The term “cyclohydrocarbyl” refers to cyclic (i.e., ring-containing) univalent radicals derived from saturated or unsaturated groups containing only carbon and hydrogen atoms, and having in the range of about 3 up to 20 carbon atoms. Exemplary cyclohydrocarbyl groups include cycloalkyl groups, cycloalkenyl groups, cycloalkadienyl groups, cycloalkatrienyl groups, cycloalkynyl groups, cycloalkadiynyl groups, spiro hydrocarbon groups wherein two rings are joined by a single atom which is the only common member of the two rings (e.g., spiro[3.4]octanyl, and the like), bicyclic hydrocarbon groups wherein two rings are joined and have two atoms in common (e.g., bicyclo[3.2.1]octane, bicyclo[2.2.1]hept-2-ene, norbomene, decalin, and the like), and the like. The term “substituted cyclohydrocarbyl” refers to cyclohydrocarbyl groups further bearing one or more substituents as set forth above.

The term “azo” refers to the bivalent group —N═N—, wherein each single bond is attached to a different carbon atom.

The term “alkylthio” refers to a straight or branched alkyl chain having from one to six carbon atoms attached to a sulfur atom. Examples of alkylthio groups include, without limitation, methylthio, ethylthio, propylthio, isopropylthio, butylthio and the like.

The term “aromatic heterocycle” refers to a stable five to seven membered ring containing one to four heteroatoms selected from oxygen, sulfur and nitrogen, and which can be fused with a benzene ring or a five to six membered ring containing from one to four heteroatoms selected from oxygen, sulfur and nitrogen. A “non-aromatic heterocycle” represents a stable four to seven membered ring containing one or two heteroatoms selected from oxygen, sulphur and nitrogen. Examples of such aromatic and nonaromatic heterocycles include thienyl, thiophenyl, furyl, oxazolyl, isoxazolyl, thiazolyl, isothiazolyl, imidazolyl, benzoturyl, benzothiophenyl, benzimidazolyl, benzoxazolyl, benzothiazolyl, indolyl, pyrrolyl, piperidinyl, pyridinyl, tetrahydrofuranyl, tetrahydropyranyl, piperazinyl, morpholinyl, thiomorpholinyl, and the like.

### Pharmaceutical Compositions

The invention further provides pharmaceutical compositions comprising a selective mGlu5 antagonist, e.g., a compound of one of formulas I-V or an enantiomer, diastereomer, N-oxide, crystalline form, hydrate, solvate, active metabolite or pharmaceutically acceptable salt thereof that is a selective mGlu5 antagonist. The pharmaceutical composition may comprise one or more excipients, such as, for example, a pharmaceutically acceptable carrier or diluent, a flavorant, a sweetener, a preservative, a dye, a binder, a suspending agent, a dispersing agent, a colorant, a disintegrant, an excipient, a lubricant, an absorption enhancer, a bactericide and the like, a stabiliser, a plasticizer, an edible oil.

Suitable pharmaceutically acceptable carriers or diluents include, but are not limited to ethanol, water, glycerol, aloe vera gel, allantoin, glycerine, vitamin A and E oils, mineral oil, phosphate buffered saline, PPG2 myristyl propionate, magnesium carbonate, potassium phosphate, vegetable oil, animal oil, and solketal.

Suitable binders include, but are not limited to: starch; gelatin; natural sugars such as glucose, sucrose and lactose; corn sweeteners; natural and synthetic gums such as acacia, tragacanth, vegetable gum, sodium alginate; carboxymethylcellulose; polyethylene glycol; waxes; and the like.

Suitable suspending agents include, but are not limited to: bentonite.

Suitable dispersing and suspending agents include, but are not limited to: synthetic and natural gums such as vegetable gum, tragacanth, acacia, alginate, dextran; sodium carboxymethylcellulose; methylcellulose; polyvinyl-pyrrolidone; and gelatin.

Suitable disintegrants include, but are not limited to: starch such as corn starch; methyl cellulose; agar; bentonite; xanthan gum; and the like.

Suitable lubricants include, but are not limited to sodium oleate, sodium stearate, magnesium stearate, sodium benzoate, sodium acetate, sodium chloride, and the like.

Suitable edible oils include, but are not limited to: cottonseed oil, sesame oil, coconut oil, and peanut oil.

Examples of additional additives include, but are not limited to sorbitol, talc, stearic acid, and dicalcium phosphate.

### Unit Dosage Forms

The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. The unit dosage forms may be used for oral, parenteral, intranasal, sublingual or rectal administration, or for administration by inhalation or insufflation, transdermal patches, and a lyophilized composition. In general, any delivery of active ingredients that results in systemic availability of such ingredients can be used. Preferably the unit dosage form is an oral dosage form, most preferably a solid oral dosage; therefore the preferred dosage forms are tablets, pills, and capsules. Parenteral preparations are also preferred.

Solid unit dosage forms may be prepared by mixing the active agents of the present invention with a pharmaceutically acceptable carrier and any other desired additives as described above. The mixture is typically mixed until a homogeneous mixture of the active agents of the present invention is obtained and the carrier and any other desired additives are formed, i.e., the active agents are dispersed evenly throughout the composition. In this case, the composition can be formed as dry or moist granules.

Tablets or pills can be coated or otherwise prepared so as to form a unit dosage form that has delayed and/or sustained action, such as controlled release and delayed release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents include, but are not limited to, polylactic acid, polyepsilon caprolactone, polyhydroxybutyric acid, polyorthoesters, polyacetals, polydihydropyrans, polycyanoacrylates and crosslinked or amphipathic block copolymers of hydrogels.

For liquid dosage forms, the active substances or their physiologically acceptable salts are dissolved, suspended or emulsified, optionally with the usually employed substances such as solubilizers, emulsifiers or other auxiliaries. Solvents for the active combinations and the corresponding physiologically acceptable salts can include water, physiological salt solutions or alcohols, e.g., ethanol, propanediol or glycerol. Additionally, sugar solutions such as glucose or mannitol solutions may be used. A mixture of the various solvents mentioned may be used in the present invention, too.

A transdermal dosage form is contemplated by the present invention as well. Transdermal forms may be a diffusion transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery systems. Transdermal dosage forms may be used for delayed release and sustained release of the active agents of the present invention.

The pharmaceutical compositions and unit dosage forms of the present invention for parenteral administration, and in particular by injection, typically include a pharmaceutically acceptable carrier, as described above. A preferred liquid carrier is vegetable oil. Injection may be, for example, intravenous, epidural, intrathecal, intramuscular, intraluminal, intratracheal or subcutaneous.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers such as targetable drug carriers. Such polymers include, but are not limited to, polyvinylpyrrolidone, pyran copolymers, polyhydroxypropylmethacrylamidophenol, polyhydroxyethylaspartamidophenol, and polyethylenoxypolylysine substituted with palmitoyl residues.

### Administration

The pharmaceutical composition or unit dosage forms of the present invention may be administered by a variety of routes, such as, without limitation, oral, enteral, intravenous, intramuscular subcutaneous, transdermal, transmucosal (including rectal and buccal) and by inhalation routes.

Preferably, the oral or transdermal route is used (i.e., with solid or liquid formulations or with skin patches, respectively).

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed. 616-638, 1986, W. B. Saunders Company, and patients affected by any physiological dysfunction related to impairment of glutamate receptor function. Such dysfunctions include, without limitation, central-nervous-system disorders such as depression, anxiety, eating disorders, sexual dysfunction, addiction and related problems.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder.

The pharmaceutical composition or unit dosage form of the present invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 to about 10 mg/kg/day and most preferably between 0.2 to about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds are formulated in capsules or tablets, preferably containing 25 to 500 mg of the compounds of the invention, and are preferably administered to a patient at a total daily dose of 25 to 1000 mg, preferably 150 to 500 mg and most preferably about 350 mg, for relief of urinary incontinence and dysfunction.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. One or more selective mGlu5 antagonist may be administered in combination with, for example, one or more antimuscarinic, α₁-adrenergic antagonist, 5-HT_{1A} receptor antagonist or COX inhibitors or NO releasing derivatives thereof. Examples of antimuscarinics, α₁-adrenergic antagonists, 5-HT_{1A} receptor antagonist, COX inhibitors and NO releasing derivatives thereof are set forth above, without limitation.

For combination treatment where the compounds are in separate dosage formulations, the compounds can be administered concurrently, or each can be administered at separate staggered times. For example, a selective mGlu5 antagonist may be administered in the morning and an antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### Uses-Methods for Treatment

Without wishing to be bound by theory, it is believed that administration of mGlu5 receptor antagonists prevents unwanted activity of the sacral reflex and/or cortical mechanisms that control micturition. Thus, it is contemplated that a wide range of neuromuscular dysfunctions of the lower urinary tract can be treated using the compounds of the present invention, including without limitation: dysuria, incontinence and enuresis (overactive bladder). Dysuria includes urinary frequency, nocturia, urgency, reduced urinary compliance (reduced bladder storage capacity), difficulty in emptying the bladder, i.e., a suboptimal volume of urine is expelled during micturition. Incontinence syndromes include stress incontinence, urgency incontinence and enuresis incontinence, as well as mixed forms of incontinence. Enuresis refers to the involuntary passage of urine at night or during sleep.

The compounds of the present invention may also be useful for the treatment of central nervous system disorders due to glutamatergic dysfunction.

### EXAMPLES

The following examples illustrate the invention as described herein, without in any way limiting it.

### Example 1

### Affinity of Selected Antagonists for mGlu Receptor Subtypes

Radioligand Binding Assay at mGlul, mGlu5 and Group II (mGlu2; mGlu3) and Group III (mGlu4; mGlu6-8) mGluRs in rat brain.

Methods

a) Membrane Preparation:

Male Sprague Dawley rats (200-300 g, Charles River, Italy) were killed by cervical dislocation and the forebrain (cortex, striatum and hippocampus) and cerebellum were homogenized (2×20 sec) in 50 vols of cold 50 mM Tris buffer pH 7.4, using a Politron homogenizer (Kinematica). Homogenates were centrifuged at 48,000×g for 15 min, resuspended in 50 vol of the same buffer, incubated at 37° C. for 15 min and centrifuged and resuspended two more times. The final pellets were frozen and stored at −80° C. until use.

b) Binding Assays:

i) mGlu1: On experimental section pellets from rat cerebellum were resuspended in 150-200 vols of 20 mM N-2-hydroxyethylpiperazine-N′-2-ethanesulfonic acid (HEPES), 2 mM MgCl₂, 2 mM CaCl₂, pH 7.4. The membranes were incubated in a final volume of 1 ml for 60 min at 25° C. with about 7.5 nM [³H]quisqualic acid in the absence or presence of competing drugs. Non-specific binding was determined in the presence of 10 μM quisqualic acid (Hinoi et al., Neurochem. Int. 38, 277-285, 2001).

ii) mGlu5: On experimental section pellets from rat forebrain were resuspended in 100 vols of 20 mM HEPES, 2 mM MgCl₂, 2 mM CaCl₂, pH 7.4. The membranes were incubated in a final volume of 1 ml for 60 min at 25° C. with 4 nM [³H]MPEP in the absence or presence of competing drugs. Non-specific binding was determined in the presence of 10 μM MPEP (Spooren et al., Trends Pharmacol Sci. 22, 331-337, 2001).

iii) Group II (mGlu2+mGlu3): On experimental section pellets from rat forebrain were resuspended in 250 vols of 20 mM HEPES, 2 mM MgCl₂, 2 mM CaCl₂, pH 7.4. The membranes were incubated in a final volume of 1 ml for 30 min at 0° C. with 1 nM [³H]LY341495 in the absence or presence of competing drugs. Non-specific binding was determined in the presence of 1 μM LY341495 (Wright et al., J. Pharmacol. Exp. Ther. 298:453-460, 2001; Mutel et al., J. Neurochem. 75, 2590-2601, 2000).

iiii) Group III (mGlu4+mGlu6+mG17+mGlu8): On experimental section pellets from rat cerebellum were resuspended in 100-200 vols of 10 mM HEPES, pH 8, 1.2 mM MgCl₂, 110 mM NaCl and 0.3 mM phenylmethylsulphonylfluoride (PMSF). The membranes were incubated in a final volume of 1 ml for 30 min at 25° C. with about 20 mM [³H]L-AP4 in the absence or presence of competing drugs. Non-specific binding was determined in the presence of 100 μM L-serine-O-phoshate (L-SOP) (Hudtloffet al., Br. J Pharmacol. 124, 971-977, 1998).

The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

Data Analysis

The inhibition of specific binding of the radioligands by the tested compounds was analyzed to estimate the inhibitory concentration 50% (IC₅₀) value by using the non-linear curve-fitting program Allfit (De Lean et al., Am. J. Physiol. 235, E97-E102, 1978). The IC₅₀ value was converted to an affinity constant (Ki) by the equation of Cheng & Prusoff (Biochem. Pharmacol. 22, 3099-3108, 1973). Data were expressed as the mean of pKᵢ(−log Ki)±SE.

Results

Table 1 shows the affinities for Group I (mGlu1, mGlu5) or Group II (mGlu2+mGlu3) receptors determined experimentally for various antagonists. Table 2 gives the values reported in the literature for the affinities and activities certain of the compounds given in Table 1 for different mGluR subtypes.

The binding of [³H]MPEP to mGlu5 subtype and of [³H]LY341495 to mGlu2+mGlu3 subtypes in rat forebrain membranes were saturable and of high affinity (K_{d}: 10.4 and 2.5 nM respectively).

The most potent inhibitors of the specific binding of [³H]MPEP to mGlu₅ receptor were MTEP and MPEP itself. SIB 1893 showed a weaker affinity, whereas all other compounds were inactive up to 1 μM, as also reported from different publications as shown in Table 2.

AIDA and LY367385 have been reported as selective antagonists of mGlu1 receptor (see Table 2). In our experimental conditions, only LY367385 significantly displaced [³H]quisqualic acid from its binding sites.

The specific binding of [³H]LY341495 to Group II (mGlu2+mGlu3) receptors was displaced with high affinity by the ligand itself whereas all other compounds where inactive up to 1 μM.

TABLE 1
Binding affinity of selected antagonists for
Group I (mGlu1, mGlu5) and Group II
(mGlu2 + mGlu3) receptor subtypes.

| | |
|---|---|
| GROUP I | GROUP II |

| | mGlu₁ | mGlu₅ | mGlu₂₊ mGlu₃ |
|---|---|---|---|
| Compound | Ki (μM) | Ki (μM) | Ki (μM) |
| MTEP | | 0.040 | |
| MPEP | >10 | 0.015 | >1 |
| SIB 1893 | >10 | 0.859 | >10 |
| AIDA | >10 | >10 | >10 |
| LY 367385 | 6 | >10 | >1 |
| LY 341495 | ≅10 | >10 | 0.004 |

TABLE 2
Binding affinities reported in the literature for mGluR subtypes.
Where radioligand binding data are not available,
IC₅₀ and pK_{b} values obtained in different
functional models are given. Values are given in μM.
ND = not determined.

| | GROUP I | | GROUP II | | GROUP III | | | |
|---|---|---|---|---|---|---|---|---|
| Compound | MGlu₁ₐ | mGlu₅ₐ | mGlu₂ | MGlu₃ | mGlu₄ₐ | mGlu₆ | MGlu₇ | mGlu₈ |
| MPEP | >100 | 0.032 | >100 | ND | >100 | >100 | >100 | >100 |
| | (IC₅₀)¹ | (IC₅₀)¹ | (IC₅₀)¹ | | (IC₅₀)¹ | (IC₅₀)¹ | (IC₅₀)¹ | (IC₅₀)¹ |
| SIB 1893 | >30 | 0.3 | >30 | ND | >30 | >30 | >30 | >30 |
| | (IC₅₀)² | (IC₅₀)² | (IC₅₀)² | | (IC₅₀)² | (IC₅₀)² | (IC₅₀)² | (IC₅₀)² |
| AIDA | 3.4 | >1000 | Weak | ND | >1000 | ND | ND | ND |
| | (pK_{b})³ | (IC₅₀)⁴ | agonist⁴ | | (IC₅₀)⁴ | | | |
| LY 367385 | 8.8 | >100 | ND | ND | ND | ND | ND | ND |
| | (IC₅₀)⁵ | (IC₅₀)⁵ | | | | | | |
| LY 341495 | 6.8 | 8.2 | 0.002 | 0.001 | 22 | ND | 0.99 | 0.173 |
| | (IC₅₀)⁶ | (IC₅₀)⁶ | (Ki)⁷ | (Ki)⁷ | (IC₅₀)⁶ | | (IC₅₀)⁶ | (IC₅₀)⁶ |

Table 2 References
¹Gasparini et al., Neuropharmacology 38: 1493, 1999.
²Varney et al., J. Pharmacol. Exp. Ther. 290: 170, 1999.
³Hermans et al., Br. J. Pharmacol. 126: 873, 1999.
⁴Moroni et al., J. Pharmacol. Exp. Ther. 281: 721, 1997.
⁵Clark et al., Biorg. Med. Chem. 7: 2777, 1997.
⁶Kingston et al., Neuropharmacology 34: 887, 1995.
⁷Johnson et al., Neuropharmacology 38: 1519, 1999.

### Example 2

### Determination of Accumulation of Inositol Phosphate

To determine the mode of action (agonist, antagonist or inverse agonist) of the test compounds at mGlu5 and mGlu 1 receptors, the concentration dependence of the stimulation of inositol phosphate production in response to glutamate is compared in the absence and presence of different concentrations of the test compounds themselves, measured in CHO-K1 cells expressing mGlu1 or mGlu5 receptors.

Determination of inositol phosphate (IP) accumulation in CHO-K1 transfected cells is performed according to Carroll et al. (Mol Pharmacol 59 : 965-973, 2001) after labelling the cells overnight with 4 μCi/ml myo-[³H]inositol. The cells are preincubated for 1 h with the glutamate-degrading enzyme (1 U/ml glutamate pyruvate transaminase) and 2 mM pyruvate to avoid the possible action of glutamate released from the cells.

The stimulation is then conducted for 30 min in a medium containing 10 mM LiCl, and different concentrations of the agonist (glutamate) or compounds to be tested. When antagonist activity is studied, test compounds are added to cell cultures 20 min prior to the addition of the agonist and further incubated in the presence of the agonist for 30 min.

The incubation is stopped by quickly washing with ice cold buffer and adding ice cold perchloric acid. Fractions of inositol monophosphates are separated from the neutralized extracts on ion exchange minicolumns.

Results are expressed as the ratio between the radioactivity collected in the IP fraction over the radioactivity recovered from the solubilized cellular membranes.

The normalized IP formation ratio is compared with that obtained with the submaximal Glu concentration used in the described experiments and referred to as 100%.

### Example 3

### Effects on Rhythmic Bladder-Voiding Contractions Induced by Bladder Filling in Anaesthetised Rats

Methods

Female Sprague-Dawley rats weighing 225-275g (Crl: CD® (SD) IGS BR, Charles River Italia) were used. The animals were housed with free access to food and water and maintained on a forced 12-hour alternating light-dark cycle at 22-24° C. for at least one week, except during the experiment. The activity on rhythmic bladder voiding contractions was evaluated according to the method of Dray (Dray J., Pharmacol. Methods, 13:157, 1985), with some modifications as in Guarneri (Guarneri, Pharmacol. Res. 27:173, 1993). Briefly, the rats were anaesthetized by subcutaneous injection of 1.25 g/kg (5 ml/kg) urethane, after which the urinary bladder was catheterized via the urethra using PE 50 polyethylene tubing filled with physiological saline. The catheter was tied in place with a ligature around the external urethral orifice and was connected to conventional pressure transducers (Statham P23 ID/P23 XL). The intravesical pressure was displayed continuously on a chart recorder (Battaglia Rangoni KV 135 with DCI/TI amplifier). The bladder was then filled via the recording catheter by incremental volumes of warm (37° C.) saline until reflex bladder-voiding contractions occurred (usually 0.8-1.5 ml). For intravenous injection of bioactive compounds, PE 50 polyethylene tubing filled with physiological saline was inserted into the jugular vein.

From the cystometrogram, the number of contractions recorded 15 minutes before (basal values) and after treatment, as well as the mean amplitude of these contractions (mean height of the peaks in mmHg), were evaluated.

Since most compounds produced an effect that was relatively rapid in onset and led to a complete cessation of bladder contractions, bioactivity was conveniently estimated by measuring the duration of bladder quiescence (i.e., the length of the time during which no contractions occurred). The effect on amplitude of bladder contractions was evaluated by comparing them (when contractions re-started) with the amplitude observed before treatment.

To compare the potency of the tested compounds for inhibiting the bladder voiding contractions, equieffective doses which resulted in the disappearance of contractions for a time of 10 minutes (ED₁₀ₘᵢₙ) were computed by means of linear regression using the least square method.

Morphine was tested, too, as a reference standard for this model.

Results

The rapid distension of the urinary bladder in urethane-anaesthetized rats produces a series of rhythmic bladder-voiding contractions whose characteristics have been described (Maggi et al., Brain Res. 380:83, 1986; Maggi et al., J. Pharmacol. Exp. Ther., 230:500, 1984). The frequency of these contractions is related to the sensory afferent arm of reflex micturition and to the integrity of the micturition centre, while their amplitude depends on the function of the reflex efferent arm. The results obtained after administration of compounds of the invention are shown in Tables 3 and 4.

The compounds of the invention selective for the mGlu5 subtype (MTEP, MPEP, SIB 1893) were found to be active as inhibitors of isovolumic voiding contractions in rats. When contractions resumed, no difference in the amplitude of the contractions was observed, relative to contractions observed before treatment.

In contrast, compounds showing selectivity for the mGlu1 subtype (AIDA, LY 367385) as well as the compound selective for the Group II mGlu receptors (LY 341495) were inactive.

In summary, the compounds of the current invention inhibited the micturition reflex by blocking bladder contractions with potency related to their affinity for the mGlu5 subtype, and in a dose-related manner. In addition, they did not reduce the amplitude of bladder contraction. The reduction effect, which can potentially cause lower bladder contractility and the undesirable retention of residual urine in the bladder after micturition, is not a characteristic of the compounds of the invention.

TABLE 3
Effects on rhythmic bladder-voiding contractions
after intravenous administration. Data represent
mean values ± SEM of the duration of
bladder quiescence (disappearance time of
contractions in min) after administration of the given doses.

| | Dose | No. | BLADDER |
|---|---|---|---|
| | (μg/ | of | CONTRACTIONS |
| COMPOUND | kg i.v.) | Rats | quiescence time (min) |
| MTEP | 30 | 5 | 5.52 ± 1.09 |
| | 100 | 6 | 8.73 ± 2.11 |
| | 300 | 6 | 13.90 ± 2.10 |
| | 1000 | 5 | 20.96 ± 4.66 |
| MPEP | 30 | 5 | 3.90 ± 1.37 |
| | 100 | 19 | 7.09 ± 1.04 |
| | 300 | 16 | 12.64 ± 1.72 |
| | 1000 | 9 | 22.46 ± 5.35 |
| SIB 1893 | 1000 | 5 | 4.44 ± 1.05 |
| | 2000 | 5 | 8.00 ± 0.78 |
| | 3000 | 6 | 15.57 ± 1.34 |
| AIDA | 1000 | 5 | 1.50 ± 0.54 |
| | 3000 | 4 | 2.33 ± 1.44 |
| | 10000 | 4 | 2.35 ± 0.50 |
| LY 367385 | 3000 | 4 | 1.00 ± 0.29 |
| | 10000 | 4 | 1.95 ± 0.30 |
| LY 341495 | 100 | 4 | 2.45 ± 1.39 |
| | 300 | 4 | 4.48 ± 1.31 |
| | 1000 | 4 | 4.65 ± 2.11 |
| Morphine | 10 | 10 | 2.76 ± 0.93 |
| | 30 | 10 | 6.89 ± 2.11 |
| | 100 | 10 | 13.83 ± 2.40 |
| | 300 | 10 | 18.17 ± 3.60 |

TABLE 4
Effects on rhythmic bladder-voiding contractions
after intravenous administration Data represent
the extrapolated dose inducing 10 min of bladder
quiescence (disappearance of contractions)
(ED₁₀ₘᵢₙ) and its 95% confidence limits.

| | ED₁₀ₘᵢₙ | | Effect on |
|---|---|---|---|
| Compound | μg/kg | 95% C.L. | amplitude |
| MTEP | 106 | 49-228 | n.a. |
| MPEP | 150 | 91-248 | n.a. |
| SIB 1893 | 1928 | 1612-2306 | n.a. |
| AIDA | >10000 | — | — |
| LY367385 | >10000 | — | — |
| LY 341495 | >1000 | — | — |
| Morphine | 50 | 28-89 | n.a. |

n.a. = not active; no significant reduction of peak heights

### Example 4

### Effect on Cystometric Parameters in Conscious Rats

Methods

Male Sprague-Dawley rats [Crl: CD® (SD) IGS BR] of 300-400 g b.w. supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 75, 1991).

Briefly, the rats were anaesthetized by intraperitoneal administration of 3 ml/kg of Equithensin solution (pentobarbital 30 mg/kg and chloral hydrate 125 mg/kg) and placed in a supine position. An approximately 10 mm long midline incision was made in the shaved and cleaned abdominal wall. The urinary bladder was gently freed from adhering tissues, emptied and then cannulated via an incision in the bladder body, using a polyethylene cannula (0.58 mm internal diameter, 0.96 mm external diameter) which was permanently sutured with silk thread. For intravenous bolus injection, a similar polyethylene tubing filled with physiological saline containing sodium heparin (40 I.U./ml) was inserted into the jugular vein. The cannulae were exteriorized through a subcutaneous tunnel in the retroscapular area, where they were connected to a plastic adapter in order to avoid the risk of removal by the animal. For drug testing, the rats were utilized one day after implantation.

On the day of the experiment, the rats were placed in modified Bollman cages, i.e., restraining cages that were large enough to permit the rats to adopt a normal crouched posture, but narrow enough to prevent turning around. After a stabilization period of about 20 minutes, the free tip of the bladder cannula was connected through a T-shaped tube to a pressure transducer (Statham P23XL) and to a peristaltic pump (Gilson minipuls 2) for continuous infusion of a warm (37° C.) saline solution into the urinary bladder, at a constant rate of 0.1 ml/minute. The intraluminal-pressure signal during infusion of saline into the bladder was continuously recorded on a polygraph (Rectigraph-8K San-ei with BM614/2 amplifier from Biomedica Mangoni) and, from the cystometrogram, two urodynamic parameters were evaluated: bladder volume capacity (BVC) and micturition pressure (MP). BVC (in ml) is defined as the volume of saline infused into the bladder necessary to induce detrusor contraction followed by micturition. MP (in mmHg) is defined as the maximal intravesical pressure caused by contraction during micturition. Basal BVC and MP values were evaluated as the mean of the values observed in the cystometrograms recorded in an initial period of 30-60 minutes. At this point in the assay, the infusion was interrupted and the test compounds were administered either intravenously by the jugular catheter or orally by a stomach tube. The bladder infusion restarted and changes in BVC and MP were evaluated from the mean values obtained in the cystometrograms observed during 1, 2, 3, 4, and 5 hours after treatment. The compounds were administered in a volume of 1 ml/kg and 2 ml/kg for intravenous and oral administration routes, respectively. Groups of control animals received the same amount of vehicle corresponding to a solution of 8% dimethylformamide and 8% Tween 80 in water (final concentrations) for intravenous, or a solution of 0.5% methocel in water for oral route.

Under the given test conditions, measurement of BVC is equivalent to measurement of interval time between micturitions.

### Statistical Analysis

All data were expressed as mean±standard error. The percent changes of BVC and MP relative to the basal values, as well as Δ values (difference in ml or in mmHg) of BVC and MP (BVC or MP at time “x” minus basal value), were also evaluated for each rat/time. In the figures, data were reported as percentage changes relative to basal values.

Statistical analysis on BVC and MP values, as well as on Δ values, was performed by S.A.S./STAT software, version 6.12. The difference between vehicle and active treatment effect was evaluated on Δ values of BVC and MP, whereas the difference between the values at different times relative to basal values was evaluated on original BVC and MP data.

### Results

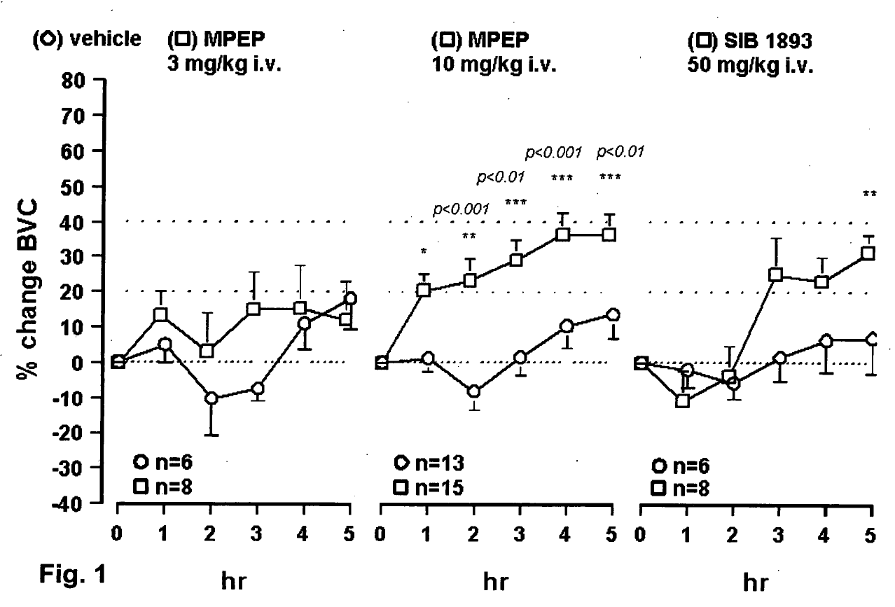

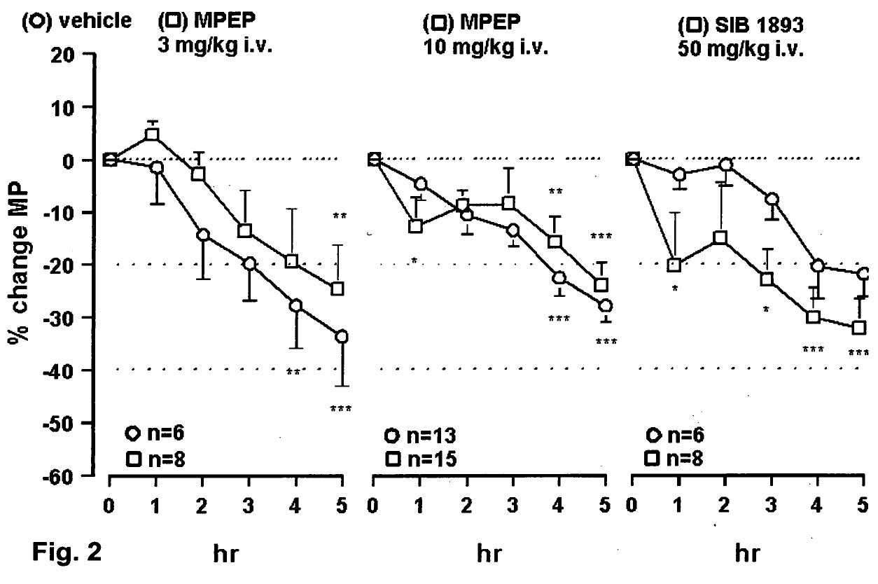

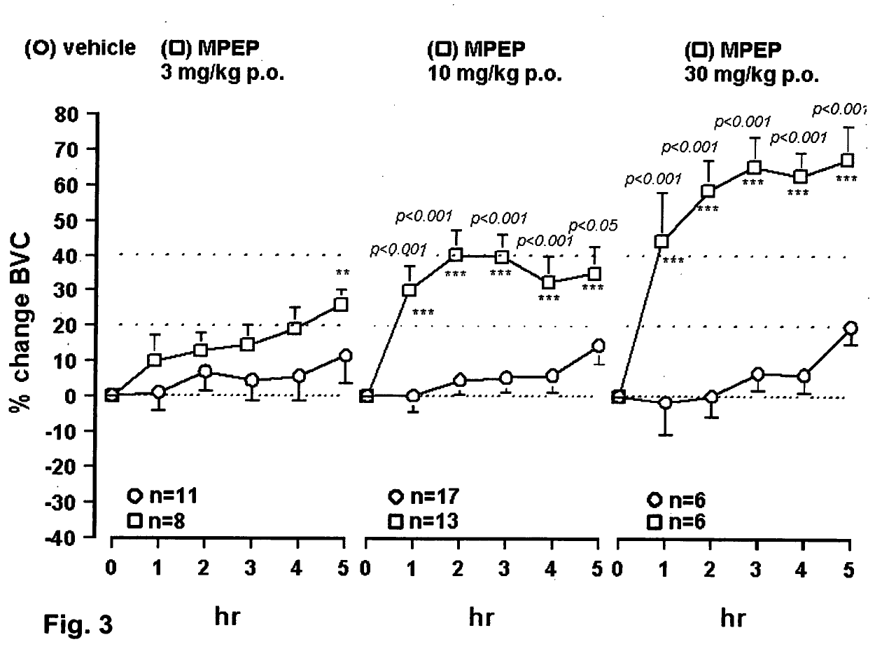

The change over time of the effects of the intravenously administered doses of the test compounds is shown in FIGS. 1 and 2. MPEP administered at a dose of 3 and 10 mg/kg i.v. and SIB 1893 administered at a dose of 50 mg/kg i.v. were found to be effective in increasing the bladder volume capacity (FIG. 1). MPEP at a dose of 10 mg/kg recorded significant increases in BVC both in comparison with the basal values and in comparison with the values recorded in the control group. SIB 1893 significantly increased the BVC only in comparison with the basal values. Both compounds induced only mild effects on the micturition pressure, with statistically significant differences versus the changes observed in the control animals (FIG. 2). MPEP administered orally at doses of 3-10 and 30 mg/kg induced significant and dose-dependent increases in the BVC at doses of 10 mg/kg or higher (FIG. 3).

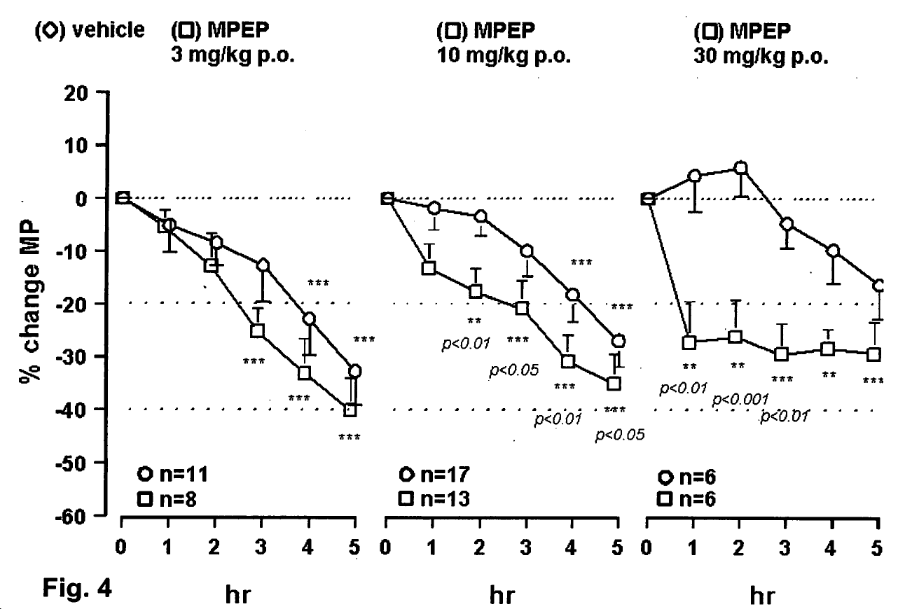

The change over time of the effects of the doses of MPEP administered orally on the MP is shown in FIG. 4. MPEP significantly reduced the MP in comparison with the values observed in the control group at doses of 10 and 30 mg/kg. The change over time of the reduction in MP was significantly different from that observed in the control group only at a dose of 30 mg/kg.

### Example 5

### Effect on Cystometric Parameters in Conscious Rats with Bladder Infused with Dilute Acetic Acid After Oral Administration

Methods

The method used was that described in Example 4. The basal BVC and MP values were evaluated as the mean of the values observed in the cystometrograms recorded over an initial period of 30-60 minutes. At this point in the test, the infusion was stopped and the test compounds were administered orally via gastric intubation. Infusion into the bladder was resumed, replacing the warm saline solution with dilute acetic acid (0.2%) and the variations in BVC and MP recorded were evaluated as mean values obtained in the cystometrograms 1, 2, 3 and 4 hours after the treatment, relative to the basal values. The compounds were administered in a volume of 2 ml/kg and the groups of control animals received the same amount of vehicle (0.5% Methocel in water) orally.

Statistical Analysis

The statistical analysis of the data was performed as reported in Example 4.

Results

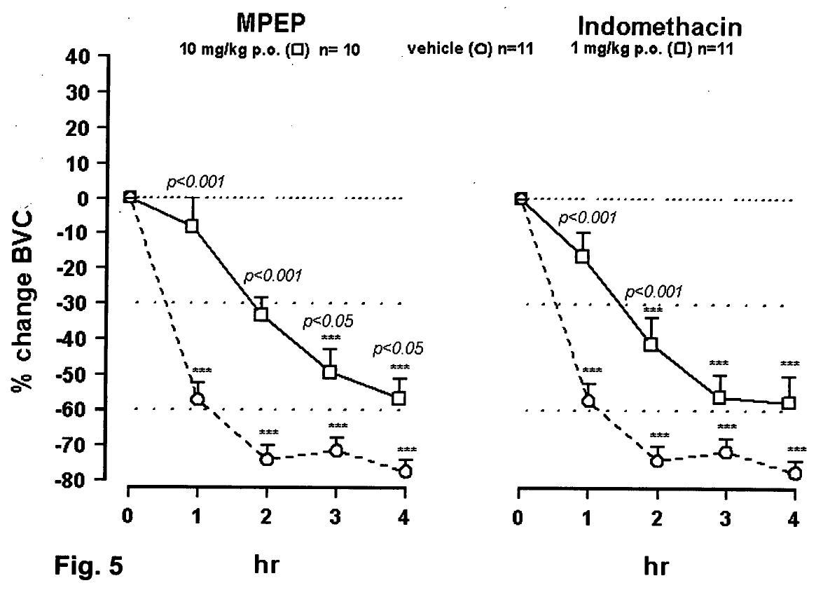

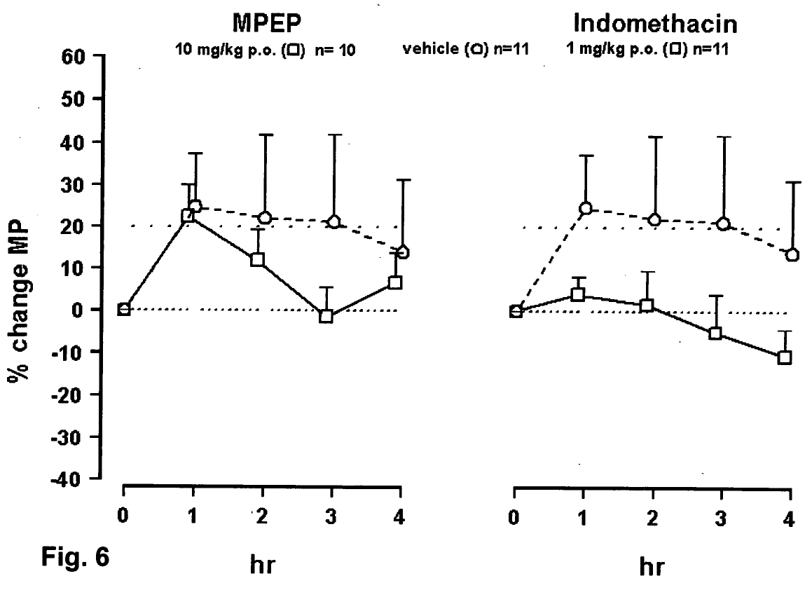

The change in time of the effects of the administered doses of the test compounds is shown in FIGS. 5 and 6. When administered orally at 10 mg/kg, MPEP was effective in antagonizing the reduction of the bladder volume capacity induced by infusing the bladder with the acid (FIG. 5), with no effect on the micturition pressure (FIG. 6). Its activity in this model was similar to that of the known antiinflammatory drug indomethacin.

### Conclusions

The mGlu receptors provide a mechanism by which glutamate can modulate or finely tune activity at the same synapses at which it elicits fast synaptic responses via ionotropic receptors. A growing body of evidence, for example, indicates that mGlu5 receptors positively modulate NMDA receptors, and that the two receptors are co-expressed in most of the neurons. This suggests that mGlu receptors represent a pharmacological target for producing relatively subtle modulation of glutamate systems in the CNS when compared to other approaches, such as non-selective NMDA receptor antagonists that produce a range of intolerable side effects in humans.

In addition, the wide diversity and heterogenous distribution of mGluR subtypes provide an opportunity for developing pharmacologic agents that selectively interact with mGluRs involved in only one or a limited number of CNS functions.

The results of the present invention clearly demonstrate that mGlu5 selective antagonists are endowed with a desirable action at the bladder level, increasing bladder capacity without negatively affecting bladder contractility. These compounds are also markedly active in the presence of bladder irritation, indicating that they can be a valid therapeutic tool in different forms of bladder overactivity.

We claim:

1. A method of treating neuromuscular dysfunction of the lower urinary tract in a mammal in need of such treat ment comprising administering to said mammal an effective amount of a compound having selective affinity for the mGlu5 subtype of the metabotropic glutamate receptors.

2. The method of claim 1 wherein said compound has an at least about 10-fold slectivity for the mGlu5 subtype of the metabotropic glutamate receptors.

3. The method of claim 1 wherein said compound has an at least about 25-fold slectivity for the mGlu5 subtype of the metabotropic glutamate receptors.

4. The method of claim 1 wherein said compound has an at least about 50-fold slectivity for the mGlu5 subtype of the metabotropic glutamate receptors.

5. The method of claim 1 wherein said compound has an at least about 100-fold slectivity for the mGlu5 subtype of the metabotropic glutamate receptors.

6. The method of claim 1 wherein said compound has an at least about 500-fold slectivity for the mGlu5 subtype of the metabotropic glutamate receptors.

7. The method of claim 1 wherein said compound is a selective mGlu5 receptor antagonist.

8. The method of claim 7 wherein said neuromuscular dysfinction is urinary urgency, overactive bladder, increased urinary frequency, decreased urinary compliance, cystitis, incontinence, urine leakage, enuresis, dysuria, urinary hesitancy or difficulty in emptying the bladder.

9. The method of claim 8 wherein said neuromuscular dysfunction that is decreased urinary compliance is decreased bladder storage capacity.

10. The method of claim 8 wherein said said neuromuscular dysfunction is interstitial cystitis.

11. The method of claim 1 wherein said compound is administered as a pharmaceutically acceptable composition.

12. The method of claim 11 wherein said compound is administered via an oral, parenteral, intranasal, sublingual, rectal or inhalatory route, or by insufflation, transdermal patches or lyophilized composition.

13. The method of claims 1 wherein said compound is administered in an amount of between about 0.01 to about 25 mg/kg/day.

14. The method of claim 13 wherein said compound is administered in an amount of between about 0.1 to about 10 mg/kg/day.

15. The method of claim 14 wherein said compound is administered in an amount of about 0.2 to about 5 mg/kg/day.

16. The method of claim 1 wherein said compound is administered at a total daily dose of about 25 to about 1000 mg.

17. The method of claim 16 wherein said compound is administered at a total daily dose of about 150 to about 500 mg.

18. The method of claim 17 wherein said compound is administered at a total daily dose of about 350 mg.

19. The method of claim 1 wherein said compound is administered in combination with an antimuscarinic drug.

20. The method of claim 19 wherein said antimuscarinic drug is selected from the group consisting of oxybutynin, tolterodine, darifenacin and temiverine.

21. The method of claim 1 wherein said compound is administered in combination with an α1-adrenergic antagonist.

22. The method of claim 21 wherein said α1-adrenergic antagonist is selected from the group consisting of prazosin, doxazosin, terazosin, alfuzosin and tamsulosin.

23. The method of claim 1 wherein said compound is administered in combination with a 5-HT_{1A} receptor antagonist.

24. The method of claim 1 wherein said compound is administered in combination with a selective COX2 inhibitor.

25. The method of claim 24 wherein said selective COX2 inhibitor comprises a NO releasing group.

26. The method of claim 1 wherein said compound is administered in combination with a non-selective COX1/COX2 inhibitor.

27. The method of claim 26 wherein said non-selective COX1/COX2 inhibitor derivative comprises a NO releasing group.

28. The method of claim 1 wherein said mammal is a human.

29. The method of claim 1 wherein said compound is administered in admixture with a pharmaceutically acceptable diluent or carrier.

30. The method of claim 29 wherein said pharmaceutically acceptable diluent or carrier is selected from the group consisting of ethanol, water, glycerol, aloe vera gel, allantoin, glycerine, vitamin A oil, vitamin E oil, mineral oil, phosphate buffered saline, PPG2 myristyl propionate, magnesium carbonate, potassium phosphate, vegetable oil, animal oil, and solketal.

31. The method of claim 1 wherein said compound having selective affinity for the mGlu5 subtype of the metabotropic glutamate receptors has a general formula I 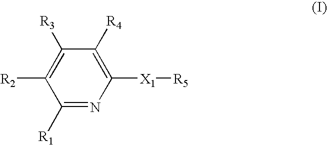
wherein:
R₁ represents hydrogen, lower alkyl, lower hydroxyalkyl, lower alkylamino, piperidino, carboxyl, esterified carboxyl, amidated carboxyl, lower alkoxy, lower haloalkyl, lower haloalkoxy, cyano, alkynyl, lower alkoxycarbonyl, di-(lower)alkylamino, lower alkylaminocarbonyl, trifluoromethylphenylaminocarbonyl or N-(lower)alkyl-N-phenylcarbamoyl, said N-(lower)alkyl and N-phenyl radicals being unsubstituted or substituted independently with a substituent selected from the group consisting of lower alkyl, lower alkoxy, halogen, and trifluoromethyl groups,
R₂ represents hydrogen, lower alkyl, carboxyl, esterified carboxyl, amidated carboxyl, lower hydroxyalkyl, hydroxyl, lower alkoxy or lower alkanoyloxy, lower alkoxycarbonyl, di-(lower)-alkylamino-(lower)alkanoyl, di-(lower)alkylaminomethyl, 4-(4-fluorobenzoyl)-piperidin-1-yl-carbonyl, 4-tert-butyloxycarbonylpiperazin-1-yl-carbonyl, 4-(4-azido-2-hydroxybenzoyl)-piperazin-1-yl-carbonyl or 4-(4-azido-2-hydroxy-3-iodo-benzoyl)-piperazin-1-yl-carbonyl,
R₃ represents hydrogen, lower alkyl, carboxy, lower alkoxycarbonyl, lower alkylcarbamoyl, lower hydroxyalkyl, di-(lower)alkylaminomethyl, morpholinocarbonyl or 4-(4-fluorobenzoyl)piperidin-1-yl-carbonyl,
R₄ represents hydrogen, lower alkyl, hydroxyl, lower hydroxyalkyl, lower aminoalkyl, (lower)alkylamino(lower)alkyl, di-(lower)-alkylamino(lower)alkyl, unsubstituted or hydroxy-substituted (lower)alkyleneamino(lower)alkyl, lower alkoxy, lower alkanoyloxy, lower aminoalkoxy, (lower)alkylamino(lower)alkoxy, di-(lower)-alkylamino(lower)alkoxy, lower alkoxycarbonyl, carboxy(lower)alkylcarbonyl, (lower)alkoxycarbonyl(lower)alkoxy, lower hydroxyalkyl, m-hydroxy-p-azidophenylcarbonylamino(lower)alkoxy, lower aminoalkoxy, phthalimido(lower)alkoxy, unsubstituted (lower)alkyleneamino(lower)alkoxy or (lower)alkyleneamino(lower)alkoxy substitued with hydroxyl or 2-oxo-imidazolidin-1-yl-groups, carboxyl, esterified carboxyl, amidated carboxyl, lower carboxyalkoxy or lower esterified carboxyalkoxy,
X₁ represents a lower alkenylene, lower haloalkenylene, lower alkynylene or lower haloalkynylene group, wherein each of the foregoing groups is linked via vicinal unsaturated carbon atoms, or an azo group (—N═N-—), and
R₅ represents an aromatic or heteroaromatic group which is unsubstituted or substituted with one or more substituents selected from lower hydroxyalkyl, lower alkoxycarbonyl, lower alkanoyl, trifluoromethyl, trifluoromethoxy, trimethylsilylalkynyl, azido, lower aminoalkoxy, di-(lower)-alkylamino(lower)alkoxy, monohalobenzylamino, thienylmethylamino, thienylcarbonylamino, trifluoromethylphenylaminocarbonyl, tetrazolyl, lower alkanoylamino, benzylcarbonylamino, (lower)alkylaminocarbonylamino, (lower)alkoxycarbonylaminocarbonylamino, (lower)alkylsulfonyl, lower alkyl, halo, lower haloalkyl, lower haloalkoxy, lower alkenyl, lower alkynyl, unsubstituted phenyl or phenyl substituted with one or more substituent selected from the group consisting of lower alkyl, lower alkoxy, halo and trifluoromethyl groups, unsubstituted phenyl(lower)alkynyl or phenyl(lower)alkynyl substituted with one or more substituent selected from the group consisting of lower alkyl, lower alkoxy, halo and trifluoromethyl groups, hydroxyl, lower hydroxyalkyl, (lower)alkanoyloxy(lower)alkyl, lower alkoxy, lower alkenyloxy, lower alkylenedioxy, lower alkanoyloxy, lower amin alkoxy, (lower)alkylamino(lower)alkoxy, (lower)alkanoylamino(lower)alkoxy, N-(lower)-alkyl-N-(lower)-alkanoylamino(lower)alkoxy, unsubstituted phenoxy or phenoxy substituted with one or more substituent selected from the group consisting of lower alkyl, lower alkoxy, halo and trifluoromethyl groups, phenyl(lower)alkoxy or phenyl(lower)alkoxy wherein the phenyl group is substituted with one or more substituent selected from the group consisting of lower alkyl, lower alkoxy, halo and trifluoromethyl groups, acyl, carboxyl, esterified carboxyl, amidated carboxyl, cyano, carboxy(lower)alkylamino, esterified carboxy(lower)alkylamino, amidated carboxy(lower)alkylamino, phosphono(lower)alkylamino, esterified phosphono(lower)alkylamino, nitro, amino, lower alkylamino, di-(lower)-alkylamino, acylamino, N-acyl-N-(lower)-alkylamino, phenylamino, phenyl(lower)alkylamino, cycloalkyl(lower)alkylamino or heteroaryl(lower)alkylamino each of which may be unsubstituted or lower alkyl- lower alkoxy-, halo- and/or trifluoromethyl-substituted,
or an enantiomer, diastereoisomer, N-oxide, crystalline form, hydrate, solvate, pharmacologically active metabolite, prodrug, or pharmaceutically acceptable salt thereof.

32. The method of claim 31 wherein said compound has a structure wherein
X₁ is a (C₂₋₄)alkenylene, (C₂₋₄)haloalkenylene, (C₂₋₄)alkynylene or (C₂₋₄)haloalkynylene group, wherein each of the foregoing groups is bonded via vicinal unsaturated carbon atoms;
R₁ is hydrogen, (C₁₋₄)alkyl, (C₁₋₄)alkoxy, hydroxy(C₁₋₄)alkyl, cyano, ethynyl, carboxy, (C₁₋₄)alkoxycarbonyl, di(C₁₋₄)alkylamino, (C₁₋₆)alkylaminocarbonyl, or trifluoromethylphenylaminocarbonyl;
R₂ is hydrogen, hydroxy, (C₁₋₄) alkyl, hydroxy (C₁₋₄) alkyl, (C₁₋₄) alkoxy, carboxy, (C₂₋₅)alkanoyloxy, (C₁₋₄)alkoxycarbonyl, di(C₁₋₄)alkylamino(C₁₋₄)alkanoyl, di(C₁₋₄)alkylaminomethyl, 4-(4-fluorobenzoyl)-piperidin-1-yl-carbonyl, 4-tert-butyloxycarbonyl-piperazin-1-yl-carbonyl, 4-(4-azido-2-hydroxybenzoyl)-piperazin-1-yl-carbonyl or 4-(4-azido-2-hydroxy-3-iodobenzoyl)-piperazin-1-yl-carbonyl;
R₃ is hydrogen, (C₁₋₄) alkyl, carboxy, (C₁₋₄)alkoxycarbonyl, (C₁₋₄)alkylcarbamoyl, hydroxy(C₁₋₄)alkyl, di(C₁₋₄)alkylaminomethyl, morpholinocarbonyl or 4-(4-fluoro-benzoyl)-piperidin-1-yl-carbonyl;
R₄ is hydrogen, hydroxy, (C₁₋₄)alkoxy, carboxy, (C₂₋₅)alkanoyloxy, (C₁₋₄)alkoxycarbonyl, amino(C₁₋₄)alkoxy, di(C₁₋₄)alkylamino(C₁₋₄)alkoxy, di(C₁₋₄)alkylamino(C₁₋₄)alkyl, carboxy(C₁₋₄)alkylcarbonyl, (C₁₋₄)alkoxycarbonyl(C₁₋₄)alkoxy, hydroxy(C₁₋₄)alkyl, di(C₁₋₄)alkylamino(C₁₋₄)alkoxy, or m-hydroxy-p-azidophenylcarbonylamino (C₁₋₄)alkoxy; and
R₅ is a group of formula 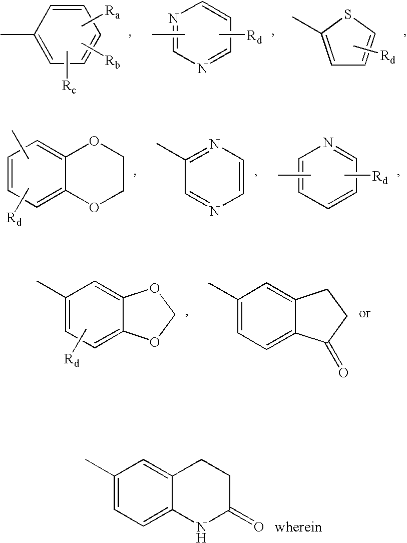
Rₐ and R_{b} independently are hydrogen, hydroxy, halogen, nitro, cyano, carboxy, (C₁₋₄)alkyl, (C₁₋₄)alkoxy, hydroxy(C₁₋₄)alkyl, (C₁₋₄)alkoxycarbonyl, (C₂₋₇)alkanoyl, (C₂₋₅)alkanoyloxy, (C₂₋₅)alkanoyloxy(C₁₋₄)alkyl, trifluoromethyl, trifluoromethoxy, trimethylsilylethynyl, (C₂₋₅)alkynyl, amino, azido, amino(C₁₋₄)alkoxy, (C₂₋₅)alkanoylamino(C₁₋₄)alkoxy, (C₁₋₄)alkylamino(C₁₋₄)alkoxy, di(C₁₋₄)alkylamino(C₁₋₄)alkoxy, (C₁₋₄)alkylamino, di(C₁₋₄)alkylamino, monohalobenzylamino, thienylmethylamino, thienylcarbonylamino, trifluoromethylphenylaminocarbonyl, tetrazolyl, (C₂₋₅)alkanoylamino, benzylcarbonylamino, (C₁₋₄)alkylaminocarbonylamino (C₁₋₄)alkoxycarbonyl-aminocarbonylamino or (C₁₋₄)alkylsulfonyl;
R_{c} is hydrogen, fluorine, chlorine, bromine, hydroxy, (C₁₋₄)alkyl, (C₂₋₅)alkanoyloxy, (C₁₋₄)alkoxy or cyano; and
R_{d} is hydrogen, halogen or (C₁₋₄)alkyl.

33. The method of claim 31 wherein said compound has a structure wherein
X₁ is a (C₂₋₄)alkenylene, (C₂₋₄)haloalkenylene, (C₂4)alkynylene or (C₂₋₄)haloalkynylene group, wherein each of the foregoing groups is linked via vicinal unsaturated carbon atoms;
R₁ is hydrogen, (C₁₋₄)alkyl, (C₁₋₄)alkoxy, cyano, ethynyl or di(C₁₋₄)alkylamino;
R₂ is hydrogen, hydroxy, carboxy, (C₁₋₄)alkoxycarbonyl, di(C₁₋₄)alkylaminomethyl, 4-(4-fluorobenzoyl)-piperidin-1-yl-carbonyl, 4-tert-butyloxycarbonyl-piperazin-1-yl-carbonyl, 4-(4-azido-2-hydroxybenzoyl)-piperazin-1-yl-carbonyl or 4-(4-azido-2-hydroxy-3-iodobenzoyl)-piperazin-1-yl-carbonyl;
R₃ is hydrogen, (C₁₋₄)alkyl, carboxy, (C₁₋₄)alkoxycarbonyl, (C₁₋₄)alkylcarbamoyl, hydroxy(C₁₋₄)alkyl, di(C₁₋₄)alkylaminomethyl, morpholinocarbonyl or 4-(4-fluoro-benzoyl)-piperidin-1-yl-carbonyl;
R₄ is hydrogen, hydroxy, carboxy, (C₂₋₅)alkanoyloxy, (C₁₋₄)alkoxycarbonyl, amino(C₁₋₄)alkoxy, di(C₁₋₄)alkylamino(C₁₋₄)alkoxy, di(C₁₋₄)alkylamino(C₁₋₄)alkyl or hydroxy(C₁₋₄)alkyl; and
R₅ is a group of formula 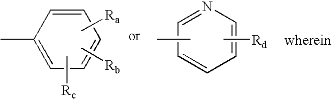
Rₐ and R_{b} independently are hydrogen, halogen, nitro, cyano, (C₁₋₄)alkyl, (C₁₋₄)alkoxy, trifluoromethyl, trifluoromethoxy or (C₂₋₅)alkynyl;
R_{c} is hydrogen, fluorine, chlorine, bromine, hydroxy, (C₁₋₄)alkyl, (C₂₋₅)alkanoyloxy, (C₁₋₄)alkoxy or cyano; and
R_{d} is hydrogen, halogen or (C₁₋₄)alkyl.

34. The method of claim 31 wherein said compound is 2-methyl-6-(phenylethynyl)pyridine (MPEP).

35. The method of claim 31 wherein said compound is 2-methyl-6-(2-phenylethenyl)pyridine (SIB 1893).

36. The method of claim 1 wherein said compound has a general formula I-A 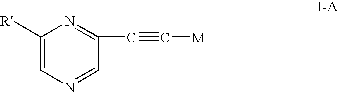
wherein
R′ is hydrogen or (C₁₋₄)alkyl and
M is a group of formula 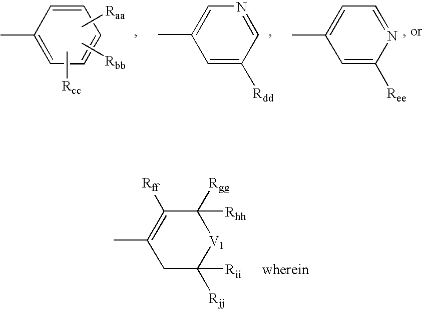
Rₐₐ, R_{bb} and R_{cc} are independently of each other hydrogen, (C₁₋₄)alkyl, (C₁₋₄)alkoxy, hydroxyl, (C₁₋₄)hydroxyalkyl, cyano or halo,
R_{dd} is cyano or halo,
Rₑₑ is hydroxyl, (C₁₋₄)alkyl or (C₁₋₄)alkoxy,
R_{ff} is hydrogen or (C₁₋₄)alkyl,
R_{gg} and Rₕₕ are hydrogen or together form a group of formula ═O, ═CH—CN, ═N—OH, ═N—O—(C₁₋₄)alkyl, ═CH—PO₃[(C₁₋₄)alkyl]₂ or ═CH—CO—Rₖₖ, wherein Rₖₖ is (C₁₋₄)alkoxy or —NRₗₗRₘₘ, where Rₗₗ and Rₘₘ are chosen independently from hydrogen, (C₁₋₄)alkyl and phenyl,
Rᵢᵢ and Rⱼⱼ are independently hydrogen, (C₁₋₄)alkyl or phenyl, and
V₁ is (CH₂)ₙ, CHRₙₙ, wherein n is 1, 2 or 3, Rₙₙ is hydroxyl, (C₁₋₄)alkyl, (C₁₋₄)alkoxy, (C₁₋₄)hydroxyalkyl, (C₁₋₄)alkoxy(C₁₋₄)alkyl, (C₁₋₄)alkoxycarbonyl, carbamoyl, (C₁₋₄)alkylcarbamoyl, phenyl, pyridyl, thienyl or (Rₒₒ, Rₚₚ)N-lower alkyl, wherein Rₒₒ is hydrogen, (C₁₋₄)alkyl, (C₁₋₄)alkanoyl or benzoyl and Rₚₚ is hydrogen or (C₁₋₄)alkyl, or, if R_{gg} and Rₕₕ are each hydrogen, V₁ can also be NR_{qq}, wherein R_{qq} is (C₁₋₄)alkoxycarbonyl, benzyloxycarbonyl, benzoyl, thienyl, (C₁₋₄)alkanoyl, carbamoyl, mono- or di-(C₁₋₄)-alkylcarbamoyl or phenylcarbamoyl, any phenyl ring in R_{qq} being optionally substituted by one or more halo, cyano, (C₁₋₄)alkyl or (C₁₋₄)alkoxy groups,
or an enantiomer, diastereoisomer, N-oxide, crystalline form, hydrate, solvate, pharmacologically active metabolite, prodrug, or pharmaceutically acceptable salt thereof.

37. The method of claim 1 wherein said compound has a general formula II-A 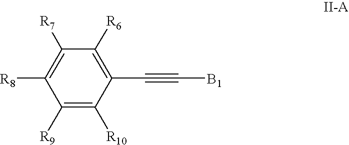
wherein
R₆, R₇, R₈, R₉ and R₁₀ represent, independently from each other, hydrogen, lower alkyl, lower alkoxy, —(CH₂)ₙ-halo, —(CH₂)ₙ—NRₑR_{f}, —(CH₂)ₙ—N(Rₑ)—C(O)-(lower)alkyl, aryl or heteroaryl, which is unsubstituted or substituted by one or more lower alkyl groups;
B₁ represents 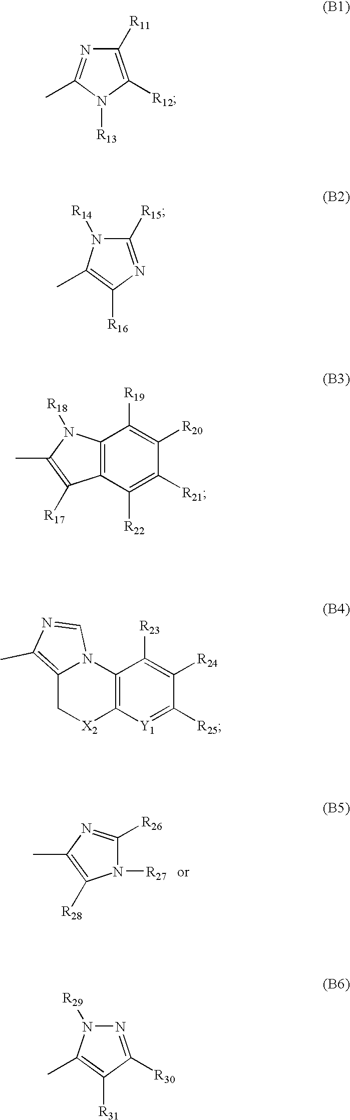
wherein
R₁₁ represents hydrogen, lower alkyl, —(CH₂)ₙ—C(O)ORₑ or halo;
R₁₂ represents hydrogen, lower alkyl, —(CH₂)ₙ—C(O)OR_{f}, halo, nitro or heteroaryl which is unsubstituted or substituted with lower alkyl or cycloalkyl;
R₁₃ represents hydrogen, lower alkyl, —(CH₂)ₙ—OH, —(CH₂)ₙ—C(O)OR_{g} or aryl;
R₁₄ represents lower alkyl;
R₁₅ represents hydrogen, lower alkyl or halo;
R₁₆ represents hydrogen or alkyl;
R₁₇ represents —(CH₂)ₙ—N(Rₑ)—C(O)-lower alkyl;
R₁₈ represents hydrogen or lower alkyl;
R₁₉, R₂₀, R₂₁ and R₂₂ represent, independently from each other, hydrogen, lower alkyl, —(CH₂)ₙ-halo or lower alkoxy;
R₂₃, R₂₄ and R₂₅ represent, independently from each other, hydrogen, lower alkyl, —(CH₂)ₙ-halo or lower alkoxy;
R₂₆ represents hydrogen or lower alkyl;
R₂₇ represents hydrogen, lower alkyl or lower alkyl substituted with one or more substituents selected from hydroxy and halo;
R₂₈ represents hydrogen, lower alkyl, lower alkanoyl or nitro;
R₂₉, R₃₀ and R₃₁ represent, independently from each other, hydrogen or lower alkyl;
Rₑ, R_{f} and R_{g} represent, independently from each other, hydrogen or lower alkyl;
n is 0, 1, 2, 3, 4, 5 or 6;
X₂ is —CH₂—, —O— or —S—; and
Y₁ is —CH═ or —N═;
or an enantiomer, diastereoisomer, N-oxide, crystalline form, hydrate, solvate, pharmacologically active metabolite, prodrug, or pharmaceutically acceptable salt thereof.

38. The method of claim 37 wherein B₁ represents B1 and R₁₂ represents (CH₂)ₙ—C(O)OR_{f}, unsubstituted heteroaryl or heteroaryl substituted with one or more lower alkyl or cycloalkyl.

39. The method of claim 38 wherein R₁₂ represents —C(O)O-lower alkyl.

40. The method of claim 1 wherein said compound has general formula II-B or II-C 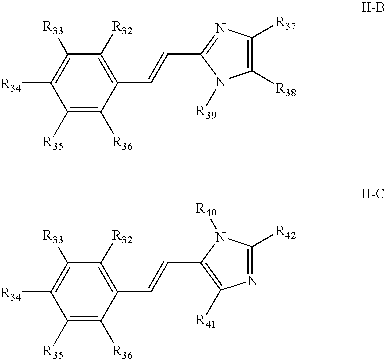
wherein
R₃₂, R₃₃, R₃₄, R₃₅ and R₃₆ represent, independently from each other, hydrogen, lower alkyl, —(CH₂)ₙ-halogen, lower alkoxy, —(CH₂)ₙ—NRₑR_{f}, —(CH₂)ₙ—N(Rₑ)—C(O)-(lower)alkyl, aryl or heteroaryl which is unsubstituted or substituted by one or more lower alkyl residues;
R₃₇ represents hydrogen, lower alkyl, —(CH₂)ₙ—C(O)ORₑ or halogen;
R₃₈ represents hydrogen, lower alkyl, —(CH₂)ₙ—C(O)OR_{f}, halogen, nitro or heteroaryl which is unsubstituted or substituted with lower alkyl or cycloalkyl;
R₃₉ represents hydrogen, lower alkyl, —(CH₂)ₙ—OH, —(CH₂)ₙ—C(O)OR_{g} or aryl;
R₄₀ represents lower alkyl;
R₄₁ represents hydrogen, halogen or lower alkyl; and
R₄₂ represents hydrogen or alkyl;
Rₑ, R_{f} and R_{g} represent, independently from each other; hydrogen or lower alkyl; and
and n=0, 1, 2, 3, 4, 5, or 6,
or an enantiomer, diastereoisomer, N-oxide, crystalline form, hydrate, solvate, pharmacologically active metabolite, prodrug, or pharmaceutically acceptable salt thereof.

41. The method of claim 1 wherein said compound has a general formula III
A₁—L₁—B₂ III
wherein
A₁ is a 5-, 6- or 7-membered ring having the structure 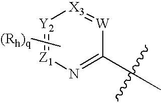
wherein
at least one of W, X₃, Y₂ and Z₁ is a group (CRₕ)ₚ, wherein p is 1 or 2; and the remainder of W, X₃, Y₂ and Z₁ are each independently O, N or S;
each Rₕ is independently, halogen, substituted or unsubstituted hydrocarbyl, substituted or unsubstituted aryl, substituted or unsubstituted heterocyclic, substituted or unsubstituted lower alkoxy, (lower)alkylcarbonyloxy, carboxyl, esterified carboxyl, amidated carboxyl, substituted or unsubstituted lower alkylthio, substituted or unsubstituted cycloalkyl, mercapto, nitro, carboxyl, carbamate, carboxamide, hydroxyl, ester, cyano, amine, amide, amidine, amido, sulfonyl, sulfonamide or N-(lower)-alkyl-N-phenylcarbamoyl wherein each nitrogen atom is independently unsubstituted or substituted independently with lower alkyl, lower alkoxy, halo or trifluoromethyl and wherein q is 0, 1, 2 or 3;
L₁ is substituted or unsubstituted alkenyl, alkynyl, or azo; and
B₂ is substituted or unsubstituted hydrocarbyl, substituted or unsubstituted cyclohydrocarbyl, substituted or unsubstituted heterocyclic, optionally containing one or more double bonds, or substituted or unsubstituted aryl,
wherein “substituted” refers to a radical wherein one or more hydrogen atoms has been replaced with a substituent selected from the group consisting of hydroxyl, alkyl, alkoxy, mercapto, aryl, heterocycle, halogen, trifluoromethyl, pentafluoroethyl, cyano, cyanomethyl, nitro, amino, N-substituted- or N,N-di-substituted amino, wherein one or both nitrogen atoms are substituted independantly with alkyl, heterocycle, aryl which are each optionally further substituted independantly with hydroxyl, alkyl or heterocycle, or, alkylamide, amidine, amido, carboxy, esterified carboxy, amidated carboxy, carboxamide, carbamate, ester, sulfonyl and sulfonamide groups, and the like,
or an enantiomer, diastereoisomer, N-oxide, crystalline form, hydrate, solvate, pharmacologically active metabolite, prodrug, or pharmaceutically acceptable salt thereof.

42. The method of claim 41 wherein said administered compound is 3-(2-methylthiazol-4-yl)ethynylpyridine (MTEP).

43. The method of claim 1 wherein said compound has a general formula IV 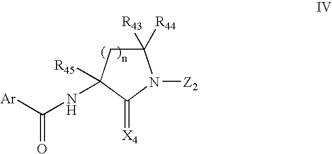
wherein,
n is 0, 1 or 2;
X₄ is O, S, NH, or NOH;
R₄₃ and R₄₄ are each independently hydrogen, CN, COORᵢ, CONHRᵢ, (C₁₋₆)alkyl, or tetrazole, or R₄₃ and R₄₄ together represent an oxo group;
Rᵢ is hydrogen or (C₁₋₆)alkyl;
R₄₅ is (C₁₋₆)alkyl, (C₂₋₆)alkenyl, (C₃₋₈)cycloalkyl, —CH₂OH, —CH₂O-alkyl, or —COOH;
Ar₁ is an unsubstituted aromatic or heteroaromatic group or an aromatic or heteroaromatic group substituted with one or more substituent selected from the group consisting of (C₁₋₆)alkylamino, di-(C₁₋₆)-alkylamino, (C₁₋₆)alkoxy, carboxy, hydroxyl, cyano, halo, trifluoromethyl, nitro, amino, (C₁₋₆)acylamino, (C₁₋₆)alkylthio, (C₁₋₆)hydroxyalkyl, (C₁₋₆)alkylsulfonyl, and (C₁₋₆)haloalkyl;
Z₂ represents a group of the formula 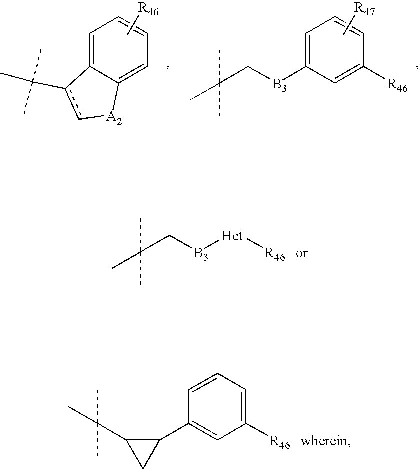
R₄₆ and R₄₇ are each independently from each other hydrogen, halogen, (C₁₋₆)alkoxy, —OAr₁, (C₁₋₆)alkyl, —CF₃, COORᵢ, CONHRᵢ, —CN, —OH, CORᵢ, —S—(C₁₋₆)-alkyl, or —SO₂-(C₁₋₆)-alkyl;
A₂ is CH₂, O, NH, NRᵢ, S, SO, SO₂, CH₂—CH₂, CH₂O, CHOH, or C(O), where Rᵢ is as defined above;
B₃ is CHRᵢ, C(Rᵢ)₂, (C₁₋₆)alkyl, C(O), —CHOH, —CH₂—O, —CH═CH, CH₂—C(O), CH₂—S, CH₂—S(O), CH₂—SO₂, —CHCO₂Rᵢ, or —CH—N(Rᵢ)₂, where Rᵢ is as defined above; and
Het is a heterocycle,
or an enantiomer, diastereoisomer, N-oxide, crystalline form, hydrate, solvate, pharmacologically active metabolite, prodrug, or pharmaceutically acceptable salt thereof.

44. The method of claim 1 wherein said compound has general formula V-A
Ar₂—G₁—Ar₃ _{V-A}
wherein
Ar₂ is a heteroaryl group,
Ar₃ is an aryl group, where
Ar₂ and Ar₃ are each independently of each other optionally substituted with one or more substituents selected from the group consisting of —F, —Cl, —Br, —I, —ORⱼ, —SRⱼ, —SORⱼ, —SO₂Rⱼ, —SO₂NRⱼRₖ, —OCORⱼ, —OCONRⱼRₖ, —NRCORₖ, —NRCO₂Rₖ, —CN, —NO₂, —CO₂Rⱼ, —CONRⱼRₖ, —C(O)Rⱼ, —CH(ORⱼ)Rₖ, —CH₂(ORⱼ), —Rⱼ, and —A—(CH₂)ₙ—NRⱼRₖ, wherein Rⱼ and Rₖ are selected independently from the group consisting of H, CF₃, (C₁₋₁₀)alkyl, cycloalkyl, alkyl-aryl, alkyl-heteroaryl, heterocycloalkyl, aryl, or Rⱼ and Rₖ may combine to form a C₁₋₅ methylene chain, and A is defined as CH₂, O, NH, S, SO, SO₂ and n is 1, 2, 3, or 4,
G₁ is selected from the group consisting of —NH—, —S, —O—, —CO—, —CONH—, —CONHCH₂—, —CH₂CONH—, —CH₂NHNH—, —CH₂NHNHCH₂—, —C═NO—CH₂—, —CH₂NHCH₂—, —CH₂CH₂NH—, —NHCH₂CO—, —NHCH₂CHOH—, —NHCH₂NHNH—, —NHCONH—, or G₁ is a cyclic group selected from the group consisting of cyclopentane, cyclopentadiene, furan, thiofuran, pyrrolidine, pyrrole, 2-imidazoline, 3-imidazoline, 4-imidazoline, imidazole, pyrazoline, pyrazolidine, imidazolidine, oxazole, 2-oxazole, thiazole, isoxazole, isothiazole, 1H-1,2,4-triazole, 1H-1,2,3-triazole, 1,2,4-oxathiazole, 1,3,4-oxathiazole, 1,4,2-dioxazole, 1,4,2-oxathiazole, 1,2,4-oxadiazole, 1,2,4-thiadiazole, 1,2,5-oxadiazole, 1,2,5-thiadiazole, 1,3,4-oxadiazole, 1,3,4-thiadiazole, 1H-tetrazole, cyclohexane, piperidine, tetrahydropyridine, 1,4-dihydropyridine, pyridine, benzene, tetrahydropyran, 3,4-dihydro-2H-pyran, 2H-pyran, 4H-pyran, tetrahydrothiopyran, 3,4-dihydro-2H-thiopyran, 2H-thiin, 4H-thiopyran, morpholine, thiomorpholine, piperazine, pyridazine, pyrimidine, pyrazine, 1,2,4-triazine, 1,2,3-triazine, 1,3,5-triazine, and 1,2,4,5-tetrazine groups,
or an enantiomer, diastereoisomer, N-oxide, crystalline form, hydrate, solvate, pharmacologically active metabolite, prodrug, or pharmaceutically acceptable salt thereof.

45. The method of claim 44 wherein Ar₃ is selected from the group consisting of phenyl, benzyl, naphthyl, fluorenyl, anthrenyl, indenyl, phenanthrenyl and benzonaphthenyl groups.

46. The method of claim 44 wherein Ar₂ is selected from the group consisting of thiazolyl, furyl, pyranyl, 2H-pyrrolyl, thienyl, pyrrolyl, imidazolyl, pyrazolyl, pyridyl, pyrazinyl, pyrimidinyl, pyridazinyl, benzothiazolyl, benzimidazolyl, 3H-indolyl, indolyl, indazolyl, purinyl quinolizinyl, isoquinolyl, quinolyl, phthalizinyl, naphthyridinyl, quinazolinyl, cinnolinyl, isothiazolyl, quinoxalinyl, indolizinyl, isoindolyl, benzothienyl, benzofuranyl, isobenzofuranyl and chromenyl groups.

47. The method of claim 44 wherein Ar₃ is selected from the group consisting of phenyl, benzyl, naphthyl, fluorenyl, anthrenyl, indenyl, phenanthrenyl and benzonaphthenyl groups and Ar₂ is selected from the group consisting of thiazolyl, furyl, pyranyl, 2H-pyrrolyl, thienyl, pyrrolyl, imidazolyl, pyrazolyl, pyridyl, pyrazinyl, pyrimidinyl, pyridazinyl, benzothiazolyl, benzimidazolyl, 3H-indolyl, indolyl, indazolyl, purinyl, quinolizinyl, isoquinolyl, quinolyl, phthalizinyl, naphthyridinyl, quinazolinyl, cinnolinyl, isothiazolyl, quinoxalinyl, indolizinyl, isoindolyl, benzothienyl, benzofuranyl, isobenzofuranyl and chromenyl groups.

48. The method of claim 1 wherein said compound has a general formula V-B 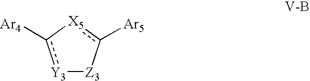
wherein
X₅, Y₃, and Z₃ are independently selected from the group consisting of N, O, S, C, and CO wherein at least one of X₅, Y₃, and Z₃ is a heteroatom;
Ar₄ and Ar₅ are independently selected from the group consisting heterocyclic and fused heterocyclic groups containing 1 to 4 heteroatoms selected from the group consisting of N, O, and S and an aromatic group selected from the group consisting of phenyl, benzyl, 1-naphthyl, 2-naphthyl, fluorenyl, anthrenyl, indenyl, phenanthrenyl, and benzonaphthenyl, wherein Ar₄ and Ar₅ are optionally substituted with one or more substituents selected from the group consisting of —F, —Cl, —Br, —I, —ORⱼ, —SRⱼ, —SORⱼ, —SO₂Rⱼ, —SO₂NRⱼRₖ, —OCORⱼ, —OCONRⱼRₖ, —NRCORₖ, —NRCO₂Rₖ, —CN, —NO₂, —CO₂Rⱼ, —CONRⱼRₖ, —C(O)Rⱼ, —CH(ORⱼ)Rₖ, —CH₂(ORⱼ)—Rⱼ, and —A—(CH₂)ₙ—NRⱼRₖ; wherein Rⱼ and Rₖ are selected independently from the group consisting of H, CF₃, (C₁₋₁₀)alkyl, cycloalkyl, alkyl-aryl, alkyl-heteroaryl, heterocycloalkyl, aryl, or Rⱼ and Rₖ may combine to form a C₁₋₅ methylene chain, A is defined as CH₂, O, NH, S, SO, SO₂,
and n is 1, 2, 3, or 4,
or an enantiomer, diastereoisomer, N-oxide, crystalline form, hydrate, solvate, pharmacologically active metabolite, prodrug, or pharmaceutically acceptable salt thereof.

49. The method of claim 48 wherein said heterocyclic or fused heterocylic group is selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl.

50. A method of identifying a compound useful for treating neuromuscular dysfunction of the lower urinary tract in a mammal, comprising
(a) determining the binding affinities of one or more test compound for an mGlu5 receptor and one or more of an mGlu1 receptor or Group II mGlu receptor;
(b) identifying a test compound that
(1) binds to mGlu5 receptor with an affinity of at least 10⁻⁶ M; and
(2) binds to mGlu5 receptor with an affinity at least 10-fold stronger than the affinity for mGlu1 receptor or Group II mGlu receptor.

51. The method of claim 50 further comprising
individually measuring the binding affinity of said one or more test compounds for one or more Group III mGlu receptor and
identifying a test compound that binds to mGlu5 receptor with an affinity at least 10-fold stronger than the affinity for a Group III mGlu receptor.

52. The method of claim 50 or 51 wherein step (b) comprises identifying a test compounds that
(1) binds to mGlu5 receptor with an affinity of at least 10⁻⁶ M; and
(2) binds to mGlu5 receptor with an affinity at least 10-fold stronger than the affinity for each of mGlu1 receptor and Group II mGlu receptor.

53. The method of claim 50 or 51 wherein step (b) comprises identifying a test compounds that
(1) binds to mGlu5 receptor with an affinity of at least 10⁻⁶ M; and
(2) binds to mGlu5 receptor with an affinity at least 100-fold stronger than the affinity for a mGlu1 receptor or Group II mGlu receptor.

54. The method of claim 53 wherein step (b) comprises identifying a test compounds that
(1) binds to mGlu5 receptor with an affinity of at least 10⁻⁶ M; and
(2) binds to mGlu5 receptor with an affinity at least 100-fold stronger than the affinity for each of mGlu1 receptor and Group II mGlu receptor.

55. The method of claim 50 or 51 further comprising measuring the ability of each of said identified test compound to act as an antagonist or inverse agonist at the mGlu5 receptor.

56. The method of claim 50 or 51 wherein said neuromuscular dysfunction is urinary urgency, overactive bladder, increased urinary frequency, decreased urinary compliance, cystitis, incontinence, urine leakage, enuresis, dysuria, urinary hesitancy or difficulty in emptying the bladder.

57. The method of claim 56 wherein said neuromuscular dysfunction that is decreased urinary compliance is decreased bladder storage capacity.

58. The method of claim 56 wherein said neuromuscular dysfunction that is cystitis is interstitial cystitis.
